(19) United States
(12) Patent Application Publication
(10) Pub. No.: US 2026/0011451 A1
(43) Pub. Date: Jan. 8, 2026

(54) SENSOR-BASED MACHINE LEARNING IN A HEALTH PREDICTION ENVIRONMENT

(71) Applicant: EVIDATION HEALTH, INC., San Mateo, CA (US)

(72) Inventor: Luca FOSCHINI, Santa Barbara (IS)

(21) Appl. No.: 19/330,712

(22) Filed: Sep. 16, 2025

Related U.S. Application Data
(63) Continuation-in-part of application No. 18/646,596, filed on Apr. 25, 2024.
Application No. 18/646,596 is a continuation of application No. 16/926,510, filed on Jul. 10, 2020, now Pat. No. 12,033,761.
(60) Provisional application No. 62/968,086, filed on Jan. 30, 2020.
(60) Provisional application No. 63/001,199, filed on Mar. 27, 2020.
(60) Provisional application No. 63/002,257, filed on Mar. 30, 2020.
(60) Provisional application No. 63/032,450, filed on May 29, 2020.

(51) Int. Cl. G16H 50/80; G06N 20/00; G16H 40/67; G16H 50/20; G16H 50/30; G16H 50/70

(52) U.S. Cl. CPC G16H 50/80; G06N 20/00; G16H 40/67; G16H 50/20; G16H 50/30; G16H 50/70

(57) ABSTRACT

A machine learning prediction system can analyze a dataset of users with self-reported symptoms and associated data from a wearable device to impact measure the impact of an acute health condition (such as the flu) at the population level. The machine learning prediction system can train a machine learning model to recognize individual acute health condition patterns based on differences in user activity with respect to the characteristics of determined baseline periods. For example, per-individual normalized change with respect to baseline aggregated at the population level can be used to determine individual acute health condition patterns and predict the onset of certain acute health conditions using a trained machine learning model. In response to predictions, the machine learning prediction system can take interventions to manage the impact of a predicted acute health condition on an individual.

### CROSS REFERENCE TO RELATED APPLICATIONS

This application is a continuation in part of U.S. application Ser. No. 18/646,596, filed Apr. 25, 2024, which is a continuation of U.S. application Ser. No. 16/926,510, filed Jul. 10, 2020, now issued as U.S. Pat. No. 12,033,761, on Jul. 9, 2024, which claims the benefit of priority of U.S. Provisional Application No. 62/968,086, filed Jan. 30, 2020, U.S. Provisional Application No. 63/001, 199, filed Mar. 27, 2020, U.S. Provisional Application No. 63/002,257, filed Mar. 30, 2020, and U.S. Provisional Application No. 63/032,450, filed May 29, 2020, each of which are incorporated herein in their entirety.

### BACKGROUND

This disclosure generally relates to managing and, in particular, to predicting or detecting the effects of an acute health condition based on physical statistic data.

Influenza-like illnesses and other acute health conditions (AHCs) can be unpredictable to the average person in both onset and recovery. For many AHCs, such as the flu, only a small percentage of events are reported to the health care system, leaving the majority of the burden of the acute health condition on society unassessed. Further, even when cases of the acute health condition are reported to the health care system, the burden in terms of lost productivity (sick days), sleep deprivation, and overall decrease in health status is unmeasured using traditional methods. Similarly, because many cases of acute health conditions are left unreported to the healthcare system, interventions or techniques to manage the impact of the acute health condition may not be adequately communicated an affected user.

A major challenge in monitoring recovery from acute or debilitating events (e.g., acute illness, surgery, or falls) is the lack of long-term individual baseline data which would enable accurate and objective assessment of functional recovery. Consumer-grade wearable devices, which may enable collection of person-generated health data (PGHD) on virtually all aspects of individual lifestyles and behaviors, may be able to provide this data.

But engagement with healthcare systems, and therefore monitoring, typically only begins when an individual is diagnosed or their symptoms otherwise become so severe that they seek care. An advantage of PGHD captured via consumer-grade wearables is that prediction or forecasting of outcomes can leverage data collected prior to the diagnosis or event, enabling early detection and treatment by “funneling” high risk individuals towards proactive screening.

Assessment of recovery may be highly challenging, primarily because canonical practice may provide no personalized baseline to which functional recovery can be compared. Equally, subjective (i.e., patient-reported) assessment of recovery may be challenging due to individual reference perceptions and expectations of what “normal” (i.e., fully recovered function) is. While evidence exists that increasing activity during rehabilitation improves recovery outcomes, triggering these interventions may be practically difficult. For example, functional recovery from lower-limb surgeries may take six months, e.g., for knee and hip replacement or hip fracture surgery. For such conditions, recovery trajectories longer than six months are typically seen as abnormal and a trigger for further intervention.

### SUMMARY

Sensor data about a user (for example, from wearable devices) can reflect physiological and behavioral changes associated with infectious disease contagion (for example, the onset of Lyme disease or influenza in a single individual) or other AHCs.

A machine learning prediction system can analyze a dataset of users with self-reported symptoms and associated data from a wearable device to measure the impact of an acute health condition (such as the flu) at the population level. For example, impact can be measured in terms of lost physical activity, increased sleep requirements, and changes in resting heart rate. Using the wearable data and acute health condition event data, the machine learning prediction system can estimate normal activity periods by learning from the dataset. For example, the machine learning prediction system can train a machine learning model on days the user is not affected by an acute health condition based on the wearable data of one or more users (or a sub-population of users) of the population.

Similarly, the machine learning prediction system can learn individual acute health condition patterns (and, in some embodiments, train a machine learning model to predict acute health condition impact) by analyzing differences in user activity with respect to the characteristics of the determined baseline periods. For example, a per-individual normalized change with respect to baseline (individual-level z-score) aggregated at the population level can be used to determine individual acute health condition patterns and predict the onset of certain acute health conditions. In response to predictions, the machine learning prediction system can take interventions to manage the impact of a predicted acute health condition on an individual (or on a group or population the individual is a member of).

Recovery for acute health conditions may be assessed relative to a personal baseline derived from long-term passive monitoring with consumer wearables. Person-generated health data (PGHD) from consumer-grade technologies can capture, and be used to predict, long-term recovery trajectories. This work may help to identify patients at risk for delayed rehabilitation early enough to trigger additional or more targeted rehabilitation interventions. Personalized recommendations based on individualized baseline data can be a major contribution of PGHD towards virtual healthcare.

There is a need for a system that can use person-generated health data from consumer-grade technologies (e.g., wearable devices) to predict a time to recovery from a debilitating event. The debilitating event may be a health condition or health intervention. The health condition may be an illness or injury. The intervention may be a surgery.

In an aspect, a method for predicting, for a subject, a recovery time from an acute or debilitating event is disclosed. The method comprises (i) retrieving wearable sensor data from a first time period and a second time period. The first time period is prior to the acute or debilitating event and wherein the second time period is after the acute or debilitating event. The method also comprises (ii) determining the recovery time for the acute or debilitating event at least in part by processing said wearable sensor data from the first time period and the second time period with a trained machine learning algorithm.

In some embodiments, the wearable sensor data comprises health measurements.

In some embodiments, the health measurements comprise at least one of sleep efficiency, step count, and heart rate.

In some embodiments, the health measurements comprise at least two of sleep efficiency, step count, and heart rate.

In some embodiments, the sensor data is collected daily throughout the first time period and the second time period.

In some embodiments, the first time period is longer than, the same length, or shorter than the second time period.

In some embodiments, the machine learning algorithm is an ensemble learning method.

In some embodiments, the machine learning algorithm uses one or more decision trees.

In some embodiments, the machine learning algorithm is random forests.

In some embodiments, the machine learning algorithm uses boosted trees.

In some embodiments, the machine learning algorithm uses gradient boosted trees.

In some embodiments, the machine learning algorithm is XGBoost.

In some embodiments, the method further comprises generating a recovery score from the wearable sensor data. Generating the recovery score comprises (i) generating a similarity group of a plurality of subjects sharing at least one characteristic with the subject, wherein the at least one characteristic relates to health data, personal data, or demographic data. Generating the recovery score also comprises (ii) calculating a ranking for the subject with respect to the similarity group. The ranking relates to (1) a type of wearable sensor data or (ii) a weighted combination of types of wearable sensor data. Generating the recovery score also comprises (iii). calculating the recovery score at least in part from the ranking.

In some embodiments, the method further comprises providing the ranking or the score to a graphical user interface (GUI).

In some embodiments, the trained machine learning algorithm is produced by: (i) maintaining, for each of a plurality of human subjects, (1) a self-reported time to recovery and (2) wearable sensor data from a first period and a second period; and (ii) training the machine learning algorithm to predict the self-reported time to recovery from the wearable sensor data.

In an aspect, a system for predicting a time to recovery from an acute or debilitating event for a subject is disclosed. The system comprises (i) a wearable device comprising one or more sensors, the one or more sensors configured to collect health data from the subject, wherein the health data is collected during a first time period and a second time period. The system also comprises (ii) a server comprising one or more processors for processing the health data from the first time period and the second time period using a machine learning algorithm. The processing produces a predicted time to recovery. The system also comprises (iii) a client device for providing the predicted time to recovery to the subject via a graphical user interface (GUI).

In some embodiments, the wearable device is a smart watch.

In some embodiments, the one or more sensors comprises at least one of a heart rate sensor, a step count sensor, or a sleep sensor.

In some embodiments, the one or more sensors comprises at least two of a heart rate sensor, a step count sensor, or a sleep sensor.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a block diagram of a system environment in which a machine learning prediction system operates, in accordance with an embodiment;

FIG. 2 is a block diagram of a machine learning prediction system, in accordance with an embodiment;

FIG. 3 is a graph illustrating example physical statistic trends for the flu, according to one embodiment;

FIG. 4 is a flowchart illustrating an example process for generating a model of an acute health condition at a machine learning prediction system, according to an embodiment;

FIG. 5 is a flowchart illustrating an example process for providing an intervention to an individual user based on predicted onset of an acute health condition for the individual user based on a model of an acute health condition, according to an embodiment;

FIG. 6 is a flowchart illustrating an example process for determining a group level intervention for a target group using a model of an acute health condition to predict AHC onset for individual users of the target group, according to an embodiment;

FIG. 7 illustrates a filtering process for an experiment to predict times to recover of human subjects;

FIG. 8 illustrates changes in activity features baseline to representative features from step, heart rate, and sleep data for periods before and after surgery;

FIG. 9 illustrates plots that show average trajectories of daily number of steps across three self-reported recovery time groups, across three lower limb surgeries;

FIG. 10 illustrates an explainable process determining features important for driving predictive power of a machine learning model;

FIGS. 11A-11F illustrate examples of a user interface from an application for reporting medical care;

FIG. 12 illustrates a plot of step data density for a plurality of patients;

FIG. 13A illustrates a four-piecewise fit used in a change point (CP) detection procedure;

FIG. 13B illustrates an example trajectory of a likelihood of a main change point;

FIG. 14 illustrates a plot showing a set of likelihood trajectories;

FIG. 15 illustrates a chart of assumed wearable PGHD availability in a set of predictive modeling experiment scenarios;

FIG. 16 illustrates a plot showing a change in a daily total number of steps pre-surgery and post-surgery;

FIG. 17A illustrates a plot showing estimated trajectories of a daily number of steps across two self-reported recovery time groups pre-surgery and post-surgery;

FIG. 17B illustrates a plot showing estimated trajectories of a daily number of steps across four self-reported recovery time groups pre-surgery and post-surgery;

FIG. 18 illustrates a system for predicting a time to recovery for a subject;

FIG. 19 illustrates a process for predicting post-procedure recovery time;

FIG. 20 illustrates screen captures of a user interface providing a score to a subject; and

FIG. 21 shows a computer system that is programmed or otherwise configured to implement methods provided herein.

### DESCRIPTION

A machine learning prediction system can utilize sensor data reflecting physiological and behavioral changes associated with an acute health condition to predict the effects of the acute health condition on individual users or a population (or population cohort) of users. An acute health condition (AHC), as used herein, is an illness, injury, or other health state associated with physiological and behavioral changes relative to a measurable baseline health state. In some embodiments, acute health conditions have a measurable onset and a recovery period before an individual who recovers returns to a baseline health state. For example, an acute health condition can be the flu (influenza) or another ILI (Influenza-Like Illness) such as H1N1 or COVID-19, another disease or illness such as a cold or Lyme disease, an acute injury, or a post-medical procedure recovery period due to a procedure that has immediate reflection on behavior or physiological signals such as total knee arthroscopy or bariatric surgery. In some implementations, a machine learning prediction system can be used to predict the effects of an acute health condition with an impact on a short enough timescale that certain portions of an individual's history can be safely assumed to not be affected by the acute health condition. For example, the assumed unaffected portions (e.g., portions of the individual's history that are not affected by the acute health condition) can be used for training a baseline model with regard to the unaffected state of the individual. More challenges can exist when predicting the impact of chronic conditions, which can go undiagnosed for a long time and may affect activity in a latent way harder to distinguish from a baseline level for if the individual was not affected by the chronic condition.

For example, a machine learning prediction system can measure the burden of the flu on a population based on retrospective data about a population of users. By learning what flu patterns look like, the system can, for instance, quantify that an average of 10,000 steps/day are lost for people affected by the flu (as a population level average), or 30 minutes of additional sleep are needed during 2+ days during the flu period, or the average RHR (resting heart rate) increases by 1 beat/minute for 3 days on average when a user is affected by the flu.

Measuring the burden of the flu (or another AHC) within a population can provide public health benefits, because only a small percentage of flu events are reported to the health care system, leaving the majority of the burden of the flu on society unassessed. Further, even when cases of the flu are reported to the health care system, the burden in terms of lost productivity (sick days), sleep deprivation, and overall decrease in health status is unmeasured using traditional methods. Additionally, flu burden measurement via activity data can be accurate and fine grained, allowing for comparative effectiveness analyses of different flu treatments (e.g., antivirals) or vaccine impact in modulating the disease in real world settings.

Once the machine learning prediction system trains a machine learning model (or simply, “model” hereinafter) of flu impact (for example, decreasing steps over 3 days of more than 3%, increase of RHR from day 2, 10+ minutes sleep lost for more than 2 days), then the system can detect the flu's onset in individuals, and potentially offer an individualized prediction or prognosis (such as of the future impact of the flu on that individual). Then, after the system learns the patterns of flu at the individual level, the machine learning prediction system can aggregate patterns to look for those patterns occurrence in real time data and gauge the state of an epidemic for example, to forecast the impact of the flu based on current data. In some implementations, the machine learning prediction system 110 predicts the onset of the flu in an individual before it occurs using a machine learning model. For example, a model can be trained to predict the probability that a user will report the onset of flu 72-120 hours in the future using activity and other contextual data (such as weather, age, and geographical location) gathered from the preceding days. In some implementations, these predictions by the machine learning prediction system are used to generate one or more interventions to manage the impact of the predicted case of the AHC. For example, the prediction might be sent to an application running on a client device of the user and/or the health care provider, and information about the prediction can be displayed in a user interface (e.g., the probability that the user will report the flu and other analysis conducted related to that probability).

### System Overview

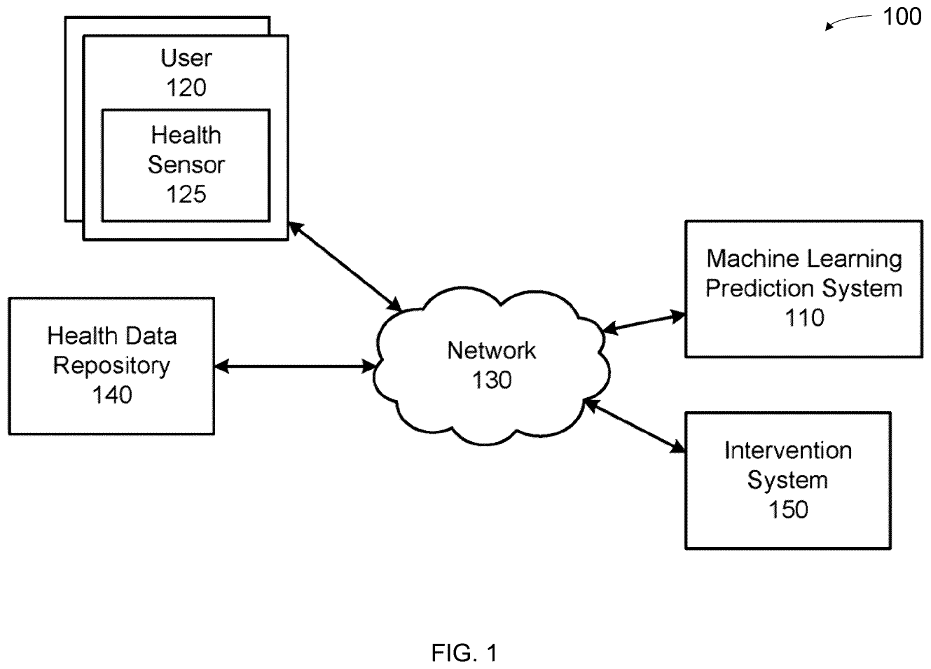

FIG. 1 is a block diagram of a system environment in which a machine learning prediction system operates, in accordance with an embodiment. The environment 100 of FIG. 1 includes a machine learning prediction system 110, a set of users 120 associated with one or more health sensors 125, a network 130, a health data database 140, and an intervention system 150.

The machine learning prediction system 110 is a server, server cluster, or cloud-based server capable of predicting the onset or impact of one or more acute health conditions on a population or individual users within a population based on physical statistics received from users 120 in the population. In some embodiments, the machine learning prediction system 110 gathers physical statistics about a set of users 120 within a population (for example, through data from one or more health sensors monitoring the physical statistics of users 120) and uses the resulting data to predict acute health condition impacts. As used herein, physical statistics are measurements characterizing a user's activity level or current health state. For example, physical statistics can include measurements of the user's vital signs such as resting heart rate (RHR), current heart rate (for example, presented as a time series), heart rate variability, respiration rate, or galvanic skin response, measurements of user activity such as daily number of steps, distance walked, time active, or exercise amount, sleep statistics such as time slept, number of times sleep was interrupted, or sleep start and end times, and/or other similar metrics. The machine learning prediction system 110 can analyze the received physical statistic data to extrapolate population level statistics about one or more acute health conditions. Similarly, the machine learning prediction system can generate AHC impact models using physical statistic data to predict acute health condition onset (or monitor acute health condition recovery) among individual users 120 within the population. In some implementations, the machine learning prediction system 110 can perform or recommend interventions on a population, population cohort, group, or individual level based on the determined population level statistics and/or predictions based on AHC impact models. The machine learning prediction system 110 will be discussed further below.

Each user 120 of the machine learning prediction system 110 is a member of a population monitored by the machine learning prediction system 110 for one or more AHCs. A user 120 can interact with health sensors 125 and the machine learning prediction system 110 through a mobile device, laptop or desktop computer, or other similar computing device. In some implementations, each user 120 is a member of one or more population cohorts characterized by demographic information, geographic location, user characteristics and/or other similar factors. A user can additionally be a member of one or more groups representing users associated with the same place of employment, attendance of a planned event, a small geographic location, or the like.

In some embodiments, each user 120 is associated with a set of health sensors 125 measuring physical statistics of that user 120. For example, the set of health sensors 125 associated with a user 120 can measure physical statistics such as the user's resting heart rate (RHR) over time, daily number of steps (and/or other measures of activity level such as distance walked or time active), and sleep statistics (such as time slept, number of times sleep was interrupted, sleep start and end times, and the like) for the user 120. The recorded physical statistics can be stored and sent by the health sensor 125 as physical statistic data can then be sent to the machine learning prediction system 110 for analysis. In some implementations, some or all physical statistic data is collected in the form of time series data consistently recording measurements of physical statistics of the user over time. The frequency of measurements included in the physical statistics data sent to the machine learning prediction system 110 can depend on the health sensor 125, user preference selections, and the type of physical statistic data being collected. For example, a health sensor 125 may send time series data for average RHR multiple times per day, but only send hours slept data once per day. In some implementations, the health sensor 125 sends physical statistic data to the machine learning prediction system 110 frequently, for example daily or in real time.

A health sensor 125 can be a wearable device or other device capable of providing physical statistics about the user 120. For example, a health sensor 125 can be a dedicated fitness tracker, a pedometer, a sleep tracker, a smart watch, smartphone, or mobile device with physical statistic monitoring functionality. For example, a health sensor 125 can be a smartphone of the user 120 with an installed physical statistic monitoring application using one or more sensors of the smartphone to measure steps, activity, movement, sleep time, or other physical statistics. An individual user 120 can be associated with multiple health sensors 125 measuring overlapping or distinct physical statistics about the user 120. The physical statistic data gathered by health sensors 125 can be sent to the machine learning prediction system 110 directly from the health sensor 125, manually uploaded to the machine learning prediction system 110 by the associated user 120 or transmitted via a third-party system to the machine learning prediction system 110. For example, the user 120 may authorize a third-party service associated with a health sensor 125 to transmit physical activity data to the machine learning prediction system 110. A user 120 or a health sensor 125 associated with a user 120 can communicate with the machine learning prediction system 110 over the network 130.

The network 130 is a network or system of networks connecting the machine learning prediction system 110 to the set of users 120 and/or health sensors 125 associated with a user 120. The network 130 may comprise any combination of local area and/or wide area networks, using wired and/or wireless communication systems. In one embodiment, the network 130 uses standard communications technologies and/or protocols. For example, the network 130 can include communication links using technologies such as Ethernet, 3G, 4G, 5G, CDMA, WIFI, and Bluetooth. Data exchanged over the network 130 may be represented using any suitable format, such as hypertext markup language (HTML) or extensible markup language (XML). In some embodiments, all or some of the communication links of the network 130 may be encrypted using any suitable technique or techniques. In some implementations, the network 130 also facilitates communication between the machine learning prediction system 110, users 120, and other entities of the environment 100 such as the health data database 140, the intervention system 150, and/or the user group system 160.

The health data repository 140 stores acute condition information about one or more users 120. For example, the health data repository 140 can be a medical provider or other entity storing records of acute health conditions. For example, a health data repository 140 can store information regarding a surgical procedure a user 120 underwent. In some implementations, users 120 can send or have sent to the machine learning prediction system 110 information about one or more acute health conditions stored in the health data repository 140. The health data repository 140 can include a server, server cluster, or other computing system with a database or other data storage system.

The intervention system 150 is a server, set of servers, server cluster, or other computing system which can perform interventions recommended by the machine learning prediction system 110. As used herein, an “intervention” is an action initiated by the machine learning prediction system 110 to reduce, mitigate, or otherwise respond to the effects of a predicted AHC on an individual or population. In some implementations, one or more interventions are directly performed by the machine learning prediction system 110, such as providing a recommendation to the user 120, however, other interventions may require additional resources or authorization not available to the machine learning prediction system 110. Only one intervention system 150 is shown in FIG. 1 for clarity, but the machine learning prediction system 110 can interface with multiple intervention systems 150 to enable performance of different interventions.

As well be discussed further below, the interventions recommended by the machine learning prediction system 110 can depend on the AHC and the specific predictions made by the machine learning prediction system 110. For example, in some implementations a machine learning prediction system 110 can predict that an individual user 120 has an AHC and recommended the intervention of a diagnostic-level test to confirm that the user 120 has the AHC. In this case the machine learning prediction system 110 can partner with an intervention system 150 that is a test provider capable of performing and determining the results of a suitable test. Similarly, an intervention system 150 can be a medical provider of the user 120 which may schedule a follow up appointment or the like to confirm the predictions of the machine learning prediction system 110 and take appropriate actions. In some embodiments, an intervention system 150 can also take population or group-level action based on a recommended intervention by the machine learning prediction system 110 based on population-level statistics or aggregated individual predictions for a group. In these embodiments, the intervention system 150 can be a government entity (such as a city or county level government entity), or a group administrator (such as an employer or an event organizer).

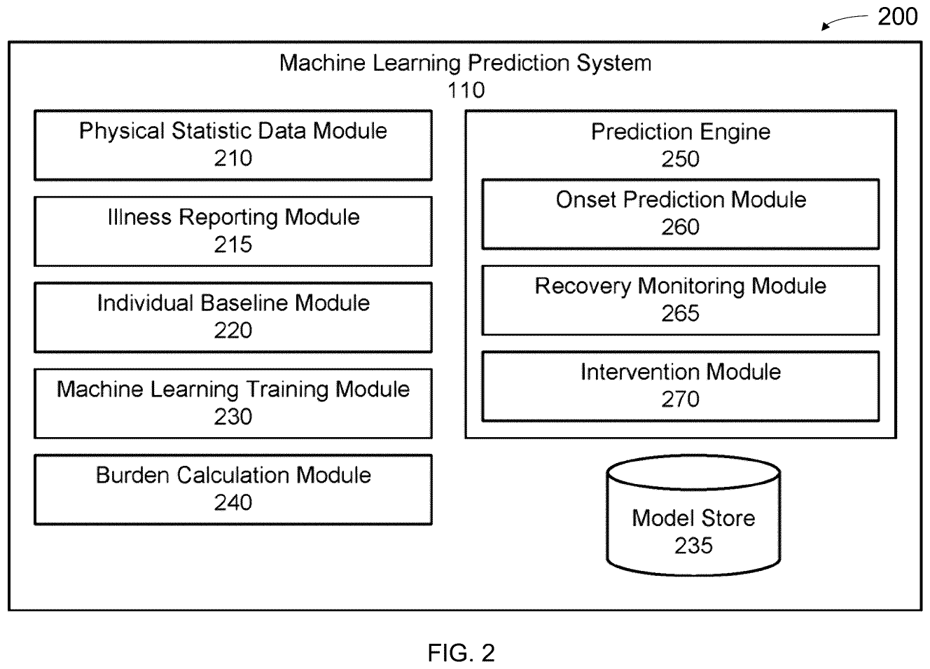

FIG. 2 is a block diagram of a machine learning prediction system, in accordance with an embodiment. FIG. 2 shows a machine learning prediction system 110 including a physical statistic data module 210, an illness reporting module 215, an individual baseline module 220, a model training module 230, a model store 235, a burden calculation engine 240, and a prediction engine 250. In other embodiments, the machine learning prediction system 110 may include additional, fewer, or different components for various applications. Conventional components such as network interfaces, security functions, load balancers, failover servers, management and network operations consoles, and the like are not shown so as to not obscure the details of the system architecture. In some embodiments, machine learning prediction system 110 can monitor a set of users 120 for multiple AHCs simultaneously, as described above. In some implementations, each module of the machine learning prediction system 110 can simultaneously perform its function for multiple distinct AHCs and, in some cases, gathered data (such as physical statistic data) can be applicable to the analysis for multiple AHCs affecting the same physical statistics.

### Data Gathering

The physical statistic data module 210 of the machine learning prediction system 110 can monitor a set of physical statistics about the set of users 120. In some implementations, the physical statistic data module 210 gathers time series datasets representing measures of the set of physical statistics of a user over time (“physical statistic data”). The physical statistic data module 210 can receive physical statistic data, process it for use by the machine learning prediction system 110, and store the physical statistic data. As described above, the physical statistic data for a user can include readings from one or more health sensors 125 associated with the user, however the physical statistic data module 210 can collect physical statistic data from other sources, such as by being logged or otherwise manually input by the associated user 120, from a health data repository 140, or from another similar source.

The physical statistic data module 210 can preprocess received physical statistic data prior further analysis by the machine learning prediction system 110. The machine learning prediction system 110 receives physical statistic data from multiple different types or models of health sensors 125 (or other sources) which can report physical statistic data in different formats and using different conventions. For example, the frequency of data points in received time series data can differ between physical statistic data collected from different health sensors 125 (even if both measure the same statistics). In some implementations, the physical statistic data module 210 can standardize received physical statistic data for further analysis, such as by transforming received time series data to be consistent across the set of physical statistic data and/or computing secondary physical statistic data from received physical statistic data. For example, the physical statistic data module 210 can receive physical statistic data for a user 120 including a rolling 5 minute average of heart rate measurements and activity data for a user and preprocess the data to a daily RHR, step count, time spent active, and sleep time (for example, determined based on a combination of time, heart rate, and activity data) for the user 120 for each data was received.

Each physical statistic monitored by the machine learning prediction system 110 can be affected by one or more AHCs. In some embodiments, physical statistic data collected by the physical statistic data module 210 can be used both in training models (along with symptom data gathered by the symptom data module 215) and for predicting AHC impact based on the generated models in real time or in near-real time. In some implementations, the physical statistic data module 210 continuously receives physical statistic data from health sensors 125 or users and preprocesses the physical statistic data for evaluation in real time or near-real time (for example, for predicting the onset of an AHC).

The illness reporting module 215 can collect reports of users 120 who have had an AHC. Users 120 can self-report having an AHC to the machine learning prediction system 110 and on which days they were affected by the AHC. In some implementations, the illness reporting module 215 can receive information on users who have had an AHC from another authorized source, such as a health data repository (if requested by or consented to by the user 120). In some implementations, the illness reporting module 215 sends surveys to users 120 (for example via a mobile device of the user 120) to determine if they had an AHC (and, in the case of a machine learning prediction system 110 monitoring multiple AHCs, which AHC). In some implementations, the surveys directly ask if the user 120 had a specific AHC, but surveys sent by the illness reporting module 215 can also ask about a set of symptoms characteristic of the AHC which can indicate if a user had the AHC and on which days they were affected by the symptoms. For example, the illness reporting module 215 can query users 120 as to whether they had experienced flu-like (ILI) symptoms in the preceding 14 days. Responding users 120 who had experienced symptoms were then asked to identify symptom days. Those who had not experienced symptoms were queried again two weeks later. The illness reporting module 215 can infer that users who reported symptoms had an ILI on the reported symptom days.

After determining that a user 120 had an AHC, the illness reporting module 215 can cross-reference physical statistic data for that user for the time period around and during the reported AHC. The illness reporting module 215 can label physical statistic data collected for that user 120 for the time period with an AHC event or log a separate AHC event for the user 120 those days. This data can be later used by the AHC to estimate the burden of the AHC on the population and to model (or improve a current model of) the AHC for making predictions related to the AHC. In some implementations, a user 120 self-reporting an AHC in real time (or near-real time) can trigger the machine learning prediction system 110 to monitor the recovery of the user 120 from the AHC.

### User Baseline Estimation

As described above, the machine learning prediction system 110 can determine what “normal” activity for a user 120 looks like in physical statistic data, by training a model on days the user isn't (or is presumed to not be) affected by an AHC. In some implementations, the machine learning prediction system 110 models what user physical statistic data (such as activity, sleep, and RHR) would look like for an individual user 120 over a certain period of time if the user 120 had not had an AHC in that time period. If the user 120 did report an AHC event for that period of time, this task equates to modeling the counterfactual situation in which that individual didn't have the AHC.

In some implementations, the individual baseline module 220 computes an average/standard deviation of user physical statistic data over a baseline period (during which the machine learning prediction system 110 infers the user 120 didn't report an AHC) and assume that during an AHC the user 120 would have exhibited the same physical statistic data patterns. In other implementations, the individual baseline module 220 can use one or more statistical models in which physical statistic data (such as steps, sleep, RHR) for individual user i at day j is modeled as a function of other users i′≠i similar to user i and other days j′≠j. For example, embodiments can use time series models (such as ARIMA (p, d, q)) with covariates and time-varying variables such as day-of-week or cross-sectional models using individual user-specific variables (such as demographics) to learn from similar users 120.

The individual baseline module 220 can additionally use modern machine learning models to estimate non-AHC baselines for a user 120. An individual machine learning model can be trained to estimate the activity of individual user i at day j based on all information available from other user 120 and other days, including contextual information (such as weather). Forecasting techniques based on “deep learning” can outperform classical approaches when calculating individual user 120 baselines. In some implementations, a machine learning prediction system 110 uses a model embedding of the total amount of physical statistic data and contextual data in non-AHC periods e.g., via deep autoencoder. This approach can be used to model resting heart rate (RHR) using other physical statistics (such as steps and sleep state).

### Acute Health Condition Modeling

The machine learning training module 230 generates AHC impact models that can be used by the machine learning prediction system 110 to estimate the burden of the associated AHC on a population or to predict the onset of the AHC in an individual, according to some embodiments. The generated AHC impact models can be stored in a database or other data storage system of the model store 235 for retrieval and use by other parts of the machine learning prediction system 110.

An AHC impact model can predict the impact of a target AHC based on physical statistic data for a defined set of physical statistics for the AHC impact model. In some implementations, an AHC impact model can be associated with a specific population or population cohort of users 120 selected by demographic information, geographic location, or other similar factors. The machine learning training module 230 can train multiple AHC impact models for the same target AHC, with each AHC impact model having a different combination for applicable population and physical statistics. For example, the machine learning training module 230 can construct an AHC impact model using a combination of RHR, step count, and sleep amount to predict the onset of an ILI. Using multiple categories of gathered wearable activity data in the same AHC impact model can provide better prediction of AHC onset, according to some embodiments. For example, a certain minority of users 120 in a given population can exhibit opposite to trend behavior in one or more physical statistics (for example, a minority of users may experience a drop in RHR after ILI onset as opposed to an increase in nRHR as can be expected based on an average user). In some implementations, the machine learning prediction system 110 uses AHC impact models combing physical statistic data in multiple categories to predict AHC onset not only to generally improve accuracy, but to account for situations where one or more categories of wearable activity may not align with expected trends for the AHC.

An AHC impact model can take recent physical statistic data for a user (for example, containing physical statistics from the current day and 3 previous days) and return a probability that a user 120 is experiencing the AHC for each of one or more days. An AHC impact model can provide a probability that the user has the AHC on the current day (or the most recent day physical statistic data is available for the user 120), on a specific future day (such as if the user 120 is likely to begin experiencing AHC symptoms tomorrow), or had the AHC on a previous day (such as if the (PSD indicates the user 120 began experiencing symptoms yesterday). In other embodiments, an AHC impact model can be trained to return a probability that the has or will imminently have the AHC, for example, based on the probability that the user has the AHC on one or more days. Similarly, AHC impact models can be trained to monitor recovery from an AHC, returning a probability a user 120 has recovered from the AHC and/or a probability the user 120 has not recovered from the AHC.

To train an AHC impact model, the machine learning training module 230 can assemble a training data set based on gathered physical statistic data and AHC event data from the illness reporting module 215. In some implementations, the machine learning training module 230 first assembles an initial training set of users 120 associated with the target AHC. A user can be selected for the training set if the user's stored physical statistic data is associated with an AHC event flag for the target AHC (for example, placed there based on the user's response to a survey from the illness reporting module 215). For example, when assembling a training data set for an ILI, the machine learning training module 230 can select users 120 who exhibited a fever or other ILI symptoms for which the machine learning prediction system 110 has RHR data (such as from a health sensor 125 of the user) for that user 120 during the same time range as the known ILI symptoms. In some implementations, the machine learning training module 230 adds additional, healthy users 120 into the training set. This initial training data set can be further refined, for example by removing users 120 who have experienced multiple instances of an AHC within a short time frame (for example, users 120 associated with instances of different AHCs within close proximity or users 120 who appeared to have the target AHC more than one time with a short gap in between), as the impact of any one instance of an AHC might not be clear for those users. Similarly, the initial training data set can be refined by removing users 120 with incomplete or low quality physical statistic data for the time range around the AHC event, for example, when the user 120 is missing data for one or more physical statistics the model (such as if the physical statistic data associated with a user contains no RHR data) will be based on or when significant gaps exist in all gathered physical statistic data for that user 120 during the time range to be analyzed.

In some embodiments, the training data set includes all available physical statistic data for users 120 in the training data set between preset dates (for example, the months of January and February of a given year) for which the data is available. In other implementations, analysis for each user 120 in the training data is based on physical statistic data for a time range around the known “day 0” onset of the target AHC. For example, the training data set can contain date from one week before the onset of the target AHC to one week after the AHC onset. The machine learning training module 230 can select the length and type of timeframe for analysis based on any suitable factors, including the availability of physical statistic data and the target AHC (for example, how long the target AHC is expected to last can affect the length of timeframe needed to model the impacts of the AHC).

AHC impact models can be based on normalized physical statistic values relative to a baseline (expressed as a percentage or absolute difference from a determined baseline for the user 120 in that physical statistic). In some implementations, the machine learning training module 230 can calculate per-user 120 normalized baselines of each collected physical statistic (for example, normalized RHR as described above, as well as a normalized step count and a normalized sleep amount for a user) for each user in the training data set. In some implementations, an individual baseline determined by the individual baseline module 220 is used as the baseline for the AHC impact model. In other embodiments, machine learning training module 230 can calculate a baseline for each physical statistic for the AHC impact model based on the physical statistic data in the days before the labeled onset of the AHC. For example, the baseline RHR for a user can include the mean and standard deviation of that user's RHR calculated for the recorded days before an ILI onset. In some embodiments, the training data can show an increase in normalized RHR compared to the baseline RHR which is correlated with ILI symptoms (such as fever) which can be captured by an ILI impact model.

In some implementations, the machine learning training module 230 generates simple rule-based models to determine AHC onset, for example, a model that detects an ILI onset based on a target day's (i.e. today's) average normalized RHR being higher than a certain, individual-specific, threshold.

In other embodiments, the machine learning training module 230 uses machine learning techniques to train AHC impact models that take into account multiple days of context and/or more than one physical statistics to predict if a user 120 has the AHC on a given day. For example, the machine learning training module 230 can use a gradient boosting techniques to generate a machine learning model (trained on the training set of user data comprising users with a known AHC and healthy users) to predict ILI onset with greater precision than the previously described rule-based models. For example, a machine learning model can be trained to predict current ILI onset using input features of nRHR from today and three previous days (i.e. four features, each containing the nRHR for one of four individual days including today). Similarly, machine learned AHC impact models can be trained using a combination of physical statistics as input features (such as a combination of nRHR, step count, and sleep time).

The machine learning training module 230 can use neural networks, deep learning techniques, gradient-boosting techniques, or another machine learning technique or combination of machine learning techniques to generate AHC impact models based on a training data set based on user physical statistic data from the physical statistic data module 210 and AHC events collected by the illness reporting model 215. For example, the machine learning training model can use machine learning techniques such as linear support vector machine (linear SVM), boosting for other algorithms (e.g., AdaBoost), neural networks, deep learning, logistic regression, naïve Bayes, memory-based learning, random forests, bagged trees, decision trees, boosted trees, or boosted stumps in various embodiments. In some implementations, the machine learning training module can re-train one or more AHC impact models for an AHC periodically or based on additional training data (physical statistic data and/or labeled AHC events) becoming available. AHC impact models can be stored in the model store 235 for later use by the prediction engine 250. For example, the machine learning training module 230 can train a machine learning model configured to predict the onset of an AHC (or the severity of an AHC) based on time series physical statistic data for a target user from a time period leading up to the (predicted) onset of the AHC.

In some implementations, AHC impact models trained by the machine learning training module 230 can have a ROC AUC (the Area Under the Curve of the Receiver Operating Characteristic curve) which is too low for the AHC impact models to serve as a standalone diagnostic test. AHC impact models can exhibit varying ROC AUC based on the machine learning techniques used and the amount, selection, and quality of physical statistic data used as inputs to the AHC impact model. An example AHC impact model using a combination of several physical statistics can exhibit a ROC AUC of between.6 and.7 and can therefore be used as an indication of the AHC (but not a diagnosis for the AHC) to trigger one or more interventions (including a follow up diagnostic test).

### Acute Health Condition Burden Estimation

In some implementations, the burden calculation module 240 can measure the burden of an AHC on a population based on retrospective data about users 120 (for example, physical statistic data for the users 120). For example, the burden calculation module 240 can measure the burden of the flu (or another ILI) on a population based on physical statistic data for the users. By learning what patterns of flu look like, the burden calculation module 240 can, for instance, quantify that 10,000 steps/day are lost for people affected by the flu (as a population level average), or 30 minutes of additional sleep are needed during 2 or more days during the flu period, or the average RHR increases by 1 beat/minute for 3 days on average when a user is affected by the flu.

Measuring the burden of an AHC in the wild can provide public health benefits, for example, only a small percentage of flu events are reported to the health care system, leaving the majority of the burden of the flu on society unassessed. Further, even when cases of the flu are reported to the health care system, the burden in terms of lost productivity (sick days), sleep deprivation, and overall decrease in health status is unmeasured using traditional methods. Similar conditions exist for other AHCs for which the burden calculation module 240 can measure a population-level burden.

AHC burden measurement via physical statistic data can be accurate and fine grained, allowing for comparative effectiveness analyses of different treatments or strategies for dealing with the AHC. For example, the burden calculation module 240 can aid in evaluating flu treatments (e.g., antivirals) or vaccine impact in modulating the flu in real world settings. To perform a comparative effectiveness analysis, the burden calculation module 240 can utilize additional received data about treatments taken by each individual such as, medication taken and vaccine taken information for the set of users 120 (provided, for example, in surveys from the illness reporting module 215). For example, a comparative effectiveness analysis may find that one antiviral treatment is more/less effective than competitors in reducing lost physical activity, sleep deprivation, and overall quality of life due to flu impact. Similarly, a comparative effectiveness analysis can show that people who are vaccinated against the flu but end up getting the flu (as the flu vaccine has is not 100% effective at preventing the flu) get a flu that impairs their physical activity (steps), sleep, and RHR more mildly than unvaccinated users who get the flu.

As described above, in some embodiments, an individual baseline for user can be used to determine a residual from a user's normal activity during periods of self-reported AHC. In some implementations, the treatment effect of an AHC on physical statistic data is estimated as a deviation from the user's baseline predicted physical statistics. In embodiments with a strong (non-AHC) baseline, the burden calculation module 240 can subtract received physical statistic data indicating an AHC from the baseline, thus obtaining residuals which may represent the burden of flu on that individual user 120. Other embodiments of the machine learning prediction system 110 can use more sophisticated models for the residuals, in order to eliminate additional confounders either unobserved or only partially captured by the baseline model. Such residual models may include impulse priors or spline-based models that can capture the expected shape of the physical statistic data over time.

In some embodiments, the computed burden of an AHC corresponds to the Individual Treatment Effect (ITE) of the AHC on the behavior/physiology of an individual. Using calculated ITEs, the machine learning prediction system 110 can monitor how ITEs are evolving over time to detect patterns of AHC at the population level (surveillance) or provide individual-level symptom tracking and forecast to individual users 120. Similarly, the machine learning prediction system can compute Average Treatment Effects (ATEs) for users 120 in different population cohorts based on ITEs of the users within a cohort. In some embodiments, the users of a cohort are similar (for example, sharing similar demographics), and users within a cohort were exposed to different treatments (e.g., antiviral) or different preventive care measures (e.g., vaccine) for the AHC. The machine learning prediction system 110 can then estimate effectiveness of different treatments and perform comparative analyses across treatments/preventative care measures (which can later be prospectively validated). When parametric methods are used to estimate flu burden separately for these cohorts, the machine learning prediction system 110 can directly compare parameter estimates to measure differences in AHC burden, for example, in the peak or duration of the change in activity or RHR compared to baseline. In some embodiments, the machine learning prediction system uses non-parametric methods to compare the integral of the burden estimated for each cohort by taking the sum of the daily residuals (relative to predicted baseline activity) of these activity measures. Because some population cohorts have different rates of getting an AHC (for example, children and older adults have higher flu infection rates than adults 18-49 years old), causal inference techniques such as propensity score matching or use of the Augmented Inverse Probability Weighted Estimator can be used to estimate treatment ATEs for each cohort. In some implementations, the machine learning prediction system 110 gathers propensity estimates from health organizations or from flu survey data collected from users 120 of the machine learning prediction system 110.

### Acute Health Condition Prediction

As described above, the machine learning prediction system 110 can use generated AHC impact models to predict AHC onset or monitor AHC recovery in an individual based on current physical statistic data for that individual. Using the predictions, the prediction engine 250 can recommend interventions based on predicted AHC onset (or an abnormal recovery) to address or mitigate the effect of the AHC on an affected individual user 120, group, or population. The prediction engine 250 of FIG. 2 includes an onset prediction module 260, recovery monitoring module 265, and an intervention module 270.

The onset prediction module 260, according to some implementations, can predict if a user has a specific AHC in a user 120 based on an AHC impact model for that AHC and physical statistic data for the user 120. The onset prediction module 260 can operate for AHCs that have a gradual onset, or where an affected individual may not realize they have the AHC for some time (even if their physical statistics are being affected by the AHC). For example, implementations of the onset prediction model 160 can be used to predict the onset of the flu, other ILIs (such as COVID-19), or infectious illnesses where an infected individual does not show full symptoms until sometime after contracting the AHC.

The onset prediction module 260 can use AHC impact models generated by the machine learning training module 230 predict AHC onset for users with no associated symptom data (in real time or in near-real time). To predict the onset of an AHC in a user 120, the onset prediction module 260 can retrieve an AHC impact model for the AHC (for example, from the model store 235), input recent physical statistic data for the user 120 (such as physical statistic data collected by the physical statistic data module 210), and calculate a resulting probability that the user 120 has the AHC (an “AHC probability”) using the AHC impact model. In some implementations, the onset prediction module 260 sends an AHC prediction (including the AHC probability for the user 120 and, for example, details about the user 120) to the intervention module 270, which can determine if the machine learning prediction system 110 will trigger an intervention based on the prediction. The intervention module 270 and the selection of interventions will be discussed further below.

In some implementations, the onset prediction module 260 can predict AHC onset for a user 120 regularly (for example, on a daily or weekly basis), based on receiving physical statistic data for the user 120, based on an anomaly in recently received physical statistic data (for example, a RHR below a baseline RHR for the user), based on a user 120 report of a recent AHC event, in response to a request by the user 120 to check for AHC onset, or for another suitable reason. In some implementations, the frequency of automatic AHC onset predictions can be based on the specific AHC, seasonal or contextual factors, or demographic features of each user 120 (such as the age of the user 120 or a risk category of a user 120 for a specific AHC). For example, seasonal AHCs (such as the flu) can be checked more frequently during the associated seasons. Each distinct AHC monitored by the machine learning prediction system 110 can be checked by the onset prediction module 260 based on different conditions. In some implementations, users 120 can request the machine learning prediction system 110 to check for an AHC or set a preference setting affecting if and how often the machine learning prediction system 110 will check for AHC onset.

The recovery monitoring module 265 can monitor the recovery of users known to have (or have recently had) an AHC. For example, the user 120 can have scheduled surgery for which a recovery period is necessary, or the machine learning prediction system 110 receives a report that the user has an injury, illness, or other AHC (self-reported or through another source). In some implementations, the recovery monitoring module 265 uses *PDS data to determine if the user's recovery is still on track relative to a standard recovery modeled through an AHC impact model for the AHC. For example, if the effects of a surgical recovery for a certain procedure normally last a week, but if the recovery monitoring module still detects the effects after 10 days, the machine learning prediction system 110 can trigger an intervention.

Similar to the onset prediction module 260, the recovery monitoring module 265 can retrieve an AHC impact model for the AHC in question (for example, from the model store 235), input recent physical statistic data for the user 120 (such as physical statistic data collected by the physical statistic data module 210), and calculate a resulting AHC probability. AHC probabilities calculated by the recovery monitoring module 265 can represent the probability that the user 120 is experiencing complications or other delays in recovery in comparison with an expected recovery, according to some embodiments. The recovery monitoring module 265 can then send an AHC prediction including the AHC probability for the user 120 to the intervention module 270, which can determine if the machine learning prediction system 110 will trigger an intervention based on the prediction.

The intervention module 270 can recommend or automatically perform interventions based on AHC predictions made by the onset prediction module 260 and/or the recovery monitoring module 265. Interventions recommended by the intervention module 270 can notify the user of the potential AHC, mitigate the impact of the AHC on a population, treat the AHC, or provide for further diagnosis of the AHC, according to some embodiments. The interventions taken by the intervention module 270 can depend on the AHC, the user 120 (for example, based on user demographics, available information about the user 120, and/or preference settings of the user), and the AHC probability of the prediction.

In some embodiments, the intervention module 270 selects from a set of applicable interventions for each AHC prediction received from the onset prediction or recovery monitoring modules 260 and 265. Each AHC monitored by the machine learning prediction system 110 can be associated with a set of one or more possible interventions that can be initiated by the intervention module 270 in response to an AHC prediction for that AHC. A single AHC prediction can result in more than one intervention for the associated user 120. Interventions available to the intervention module 270 can be applicable across a set of different AHCs (such as sending a notification of the AHC prediction to the user 120) or specific to a single AHC (such as sending a specific diagnostic test designed to diagnose a specific AHC). Similarly, interventions can be based on if the AHC prediction was from the onset prediction module 260 or the recovery monitoring module 265 (for example, scheduling diagnostic appointment for an AHC instead of a follow up appointment for an AHC being recovered from).

In some implementations, each intervention is associated with an intervention threshold which the intervention module can use to determine if the intervention should be initiated for a given AHC prediction. For example, the intervention module 270 can compare the AHC probability of an AHC prediction to the intervention threshold for each possible intervention to determine which (if any) interventions should be initiated in response to the AHC prediction. More expensive, inconvenient, or limited (such as a doctor's appointment) can be assigned higher intervention thresholds than less costly interventions (such as a notification to the user), according to some embodiments. In addition, the intervention module 270 can select to initiate (or not initiate) an intervention based on additional factors (in combination with the AHC prediction), such as user 120 demographics or preference selections, or other selected interventions (for example, interventions may be mutually exclusive or counterproductive if performed together). In some implementations, the intervention module 270 uses intervention thresholds selected for relatively high precision to provide reasonably sure alerts. Use of an intervention threshold set for high precision may sacrifice recall (for example, only predicting ˜10% of the true cases of an AHC), but an intervention that is triggered is more likely to be triggered based on an actual AHC event rather than a false positive.

Similarly, some interventions can be associated with relative intervention thresholds, where the AHC probability from an AHC predictions is ranked or otherwise compared with a set of other AHC predictions for that AHC made during the same day, week, or other defined time period to determine which AHC predictions of the set receive the intervention. For example, interventions can be selected based on a “top K” threshold where the K AHC predictions with the highest AHC probabilities receive the intervention or a “top %” threshold where a percentage of the AHC predictions with the highest AHC probability receive the intervention. Relative intervention thresholds can be useful for interventions which may have a limited supply due to logistical constraints, cost, or limited availability. In some implementations, relative intervention thresholds are used in combination with other intervention thresholds, such as an intervention selecting the top 150 AHC predictions with an AHC probability above 0.9 to receive the intervention.

As described above, the intervention module can initiate interventions that are carried out by (or partially by) an intervention system 150 separate from the user 120 and the machine learning prediction system 110. The intervention system associated with each initiated intervention can differ based on the selected intervention and the user 120 receiving the intervention. For example, if the intervention is to schedule a doctor's appointment, the intervention system 150 can be a specific doctor of the user 120.

### AHC Applications

In some implementations, the set of interventions associated with an AHC can include one or more messages sent to the user 120 notifying the user of the AHC prediction and including instructions to mitigate the effects of the AHC, instruct the user how to prevent the AHC, and/or reduce the spread of the AHC depending on the properties of the AHC. For example, the intervention module 270 can send a message to a user 120 instructing the user to practice social distancing techniques or other spread-minimizing actions. If the AHC is the flu (or COVID-19 or another ILI) such a message might read “you look like you may be coming down with the flu, consider minimizing contact, here's a link to CDC guidelines.” In cases of an infectious AHC (such as COVID-19 or an ILI), the intervention message can instruct the user 120 to test themselves and input the results to a contact tracing resource (such as a contact tracing application) and/or to check the contact tracing resource to see if they have been exposed to the AHC.

As described above, the intervention module 270 can also recommend or automatically schedule doctor appointments on predicting AHC onset. In some implementations, the intervention module 270 can interface with a doctor's office or other healthcare provider (as an intervention system 150) to schedule or recommend scheduling a doctor's appointment for the associated user 120. For example, some users 120 can be diagnosed with an AHC at a point of care (such as a hospital or doctor's office) but sent home for treatment due to low severity of current symptom manifestations. The machine learning prediction system 110 can be used to monitor those users 120 for more severe symptoms occurring. In some implementations, a specifically trained AHC impact model can output a “probability of symptoms worsening” for this use case. On detecting more severe symptoms, the intervention module 270 can inform the user and/or point of care and, in some implementations, schedule an appointment at the point of care.

Similarly, the machine learning prediction system 110 can also monitor users 120 receiving specific treatments for an AHC (including experimental treatments or treatments with a risk of adverse effects). On detecting a worsening of symptoms (or other adverse effects) the intervention module 270 can notify a healthcare provider administering the treatment (or other relevant entity) to follow up with the user 120 and determine if adverse effects are experienced (and if they are related to the treatment). For example, developing vaccines requires specific monitoring of drug safety and effectiveness for which the machine learning prediction system 110 can be used to identify adverse effects in users 120 receiving the vaccine.

In some embodiments, the machine learning prediction system 110 can be used to monitor for symptoms of an AHC and schedule a diagnostic test for the AHC at a healthcare provider and/or have an at-home test sent to the user 120. Using the machine learning prediction system 110 to select users 120 to receive tests can be effective, even if implemented over a long time period (in contrast to relying on daily symptom polling of users 120, which will experience reporting fatigue and a potentially a loss in effectiveness over time) and provide higher probability of finding cases of the AHC than randomly selecting users 120 to test. In cases where there is a limited supply of tests (not adequate to cover all users potentially experiencing the AHC), using the machine learning prediction system 110 to triage users 120 to be sent tests can magnify the impact of the available tests in finding cases of the AHC. This application of the machine learning prediction system 110 will be discussed further below.

The intervention module 270 can also pool AHC predictions collected for users 120 belonging to a population cohort or group of users to take actions on a group level. For example, a subset of users 120 can be associated with an employer, large event (such as a conference, festival, or the like), or other large gathering of users 120 based on approved association by the user 120 or the like. For an infectious AHC, the intervention module 270 can aggregate each AHC prediction for users 120 associated with the group to determine an approximate rate of infection (or estimated number of users infected with the AHC) within the group based, for example, on the AHC probability associated with AHC predictions of users 120 in the group. If the approximate rate of infection (of the group) reaches an intervention threshold, the intervention module can notify an authority of the group (such as an employer, event organizer, or local government) of the approximate rate of infection (while not notifying the group authority of any specific (AHC predictions of users 120) and/or recommend other actions such as canceling large events, implementing spread-minimization policies at an office building, or the like.

Similarly, users 120 can be grouped based on geographic area and estimated rate of infection/number of infected users 120 can be aggregated by the intervention module 270 based on geographic groups. In some implementations, the geographic groups can be separated based on county, municipality, neighborhood, or other similar geographic designation. Using these groups, the machine learning prediction system 110 can detect “hotspots” of an infections AHC such as COVID-19 or the flu and transmit this information to local health authorities, which can inform decisions on public health policy, or easing/implementing restrictions due to the AHC. In some implementations, using the machine learning prediction system 110 predictions based on current data provides a timing advantage over delayed signals such as “number of hospitalizations” (which can result in significant lag time before a patient becomes sick enough to require hospitalization) when making policy changes.

### Example AHC Application: Testing Triage

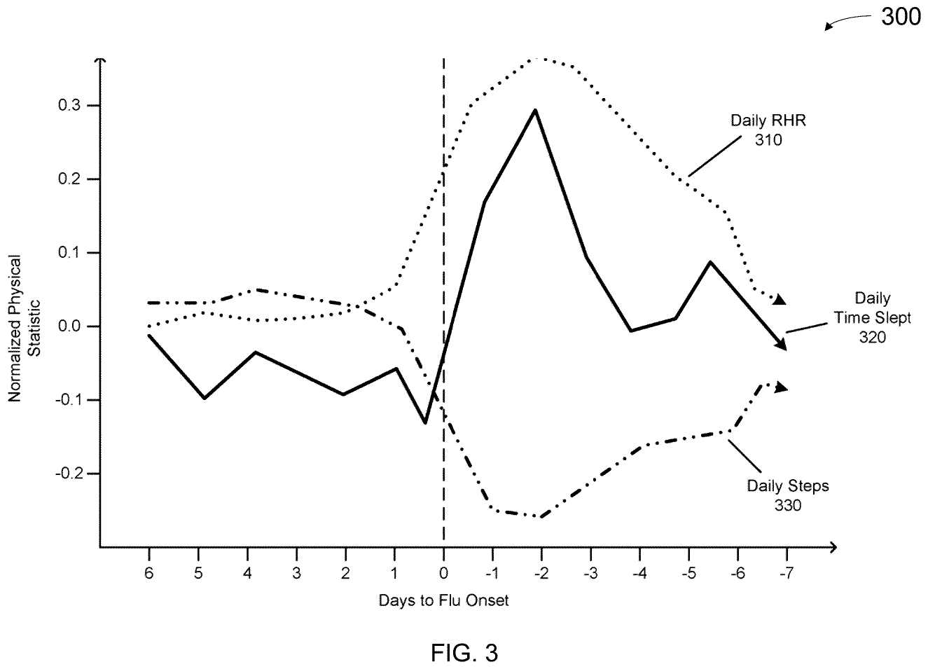

FIG. 3 is a graph illustrating example physical statistic trends for the flu (or another ILI), according to one embodiment. The graph 300 of FIG. 3 shows the physical statistic data for normalized daily RHR 310, daily time slept 320, and daily steps 330 organized according to days until onset of the flu (when the user 120 would recognize flu symptoms). The graph 300 shows example trends in physical statistics for users 120 experiencing the flu (an AHC) that can be determined by the machine learning prediction system 110 based on user 120 *PDS data and reported AHC events (such as flu symptom reports) gathered by the illness reporting module 215. Trends detected for other AHCs (or even other ILIs) can exhibit different trends. Similarly, physical statistics other than RHR 310, time slept 320, and daily steps 330 can also exhibit trends for the flu, other ILIs, or other AHCs.

The graph 300 shows trend lines for daily RHR 310, daily time slept 320, and daily steps 330 from 6 days before flu onset to 7 days after flu onset. In this example, the daily RHR trend 310 and daily time slept trend 320 show increases in normalized RHR and time slept starting prior to flu onset. Similarly, the daily steps trend 330 shows a decrease in the day prior to flu onset. Therefore, daily RHR, time slept, and steps can be used as physical statistics for predicting onset of the flu or another ILI, according to some embodiments. Appendix A, a paper titled “Measuring COVID-19 and Influenza in the Real World via Person-Generated Health Data” discloses further details about physical statistic trends for COVID-19 compared to non-COVID-19 flu or other ILIs and is hereby incorporated by reference. For example, COVID-19 may cause a measurable increase in RHR (compared to a baseline RHR) in the days surrounding AHC onset.

In some situations (such as an outbreak of an infectious ILI like COVID-19), large amounts of tests on users 120 of a population can be performed to mitigate the spread of the outbreak, for example to recognize and contain infected clusters. For example, “testing at home” technology can be used to mail test kits to user 120 to take a diagnostic test for the ILI in addition to “point of care testing” performed at a healthcare facility or other specific site. However, the testing capacity available to the general population can be limited (due to logistics or availability concerns). Additionally, testing the general population can rely on members of the general population to request a test on their own initiative, which can result in some users 120 meeting guidelines for a test waiting to request a test (or just not requesting a test). Therefore, the users 120 can be aided in the process of symptom recognition and following through by technology. In some cases, users 120 can be polled daily to report their symptoms and the machine learning prediction system 110 can have tests shipped to those who report a constellation of symptoms consistent with the *ILI (such as COVID19 infection). However, over an extended period of daily polling users 120 may experience reporting fatigue and a corresponding drop in compliance/accuracy of the polling. Similarly, randomly assigning tests can result in tests on users 120 unlikely to be infected with the ILI. Therefore, the machine learning prediction system 110 can implement a “testing triage” system using a trained AHC impact model for the ILI to help determine which users 120 to be sent tests. In some implementations, as compared to randomly sampling a set of users 120 to poll every day for symptoms, the machine learning prediction system 110 system can be significantly more efficient in finding individuals that will test positive for the ILI.

As described above, the machine learning prediction system 110 can gather physical statistic data of users through the physical statistic data module 210 and augment that gathered data with symptom reports gathered by the illness reporting module 215. Using gathered training data, the machine learning module 230 can determine an AHC impact model for the ILI. Table 1 shows statistics of three AHC impact models designed to predict ILI onset based on different amounts of physical statistic data input data.

TABLE 1

| | | | | Approximate |
|---|---|---|---|---|
| | Input Data | AUROC | Precision | Gain |
| Forecasting | Days −7 | .65 | .0471 | 1.5x |
| model | to −1 | | | |
| Nowcasting | Days −7 | .67 | .0644 | 2x |
| model | to 0 | | | |
| Detection | Days −7 | .69 | .0979 | 3x |
| model | to 3 | | | |

Table 1 includes statistics for a “forecasting model,” a “nowcasting model,” and a “detection model,” with needed input data, AUROC, precision, and an approximate gain relative to random testing for each. In some implementations, testing can be assigned to users 120 based on a “top K” intervention threshold (as describe above) to select a certain number of users the model is most confident are experiencing the ILI to receive the test. The approximate gain is calculated based on a ratio of precision of the model over prevalence of the ILI in the sample set. The approximate gain can measure an advantage of the model over a randomized testing strategy in finding users 120 infected with the ILI.

The forecasting model shown in table 1 takes input for the 7 days prior to the target day (for example, if using current data, the forecasting model would predict an ILI onset tomorrow). Out of the three example models, the forecasting model is the least accurate, but provides the most advance notice in onset prediction (and still provides a 1.5× gain, depending on the chosen top K threshold). The nowcasting model takes input for the 7 days prior to the target day and the target day itself (for example, if using current data, the forecasting model would predict an ILI onset today). Finally, the detection model uses data for the 7 days prior to the target day, the target day itself, and three days after the target day (for example, if using current data, the forecasting model would predict an ILI onset three days ago). The example detection model shows the best AUROC, precision, and gain, but requires data after the target day prediction.

Implementations of a testing triage system can select an AHC impact model (or a combination of models) based on the properties of the ILI, the test for the ILI, or the testing capacity of the system. For example, if testing capacity is severely limited a model that produces a higher gain might be chosen, but if quick identification of potential cases is the main concern a forecasting model can be chosen.

### Machine Learning Prediction System Processes

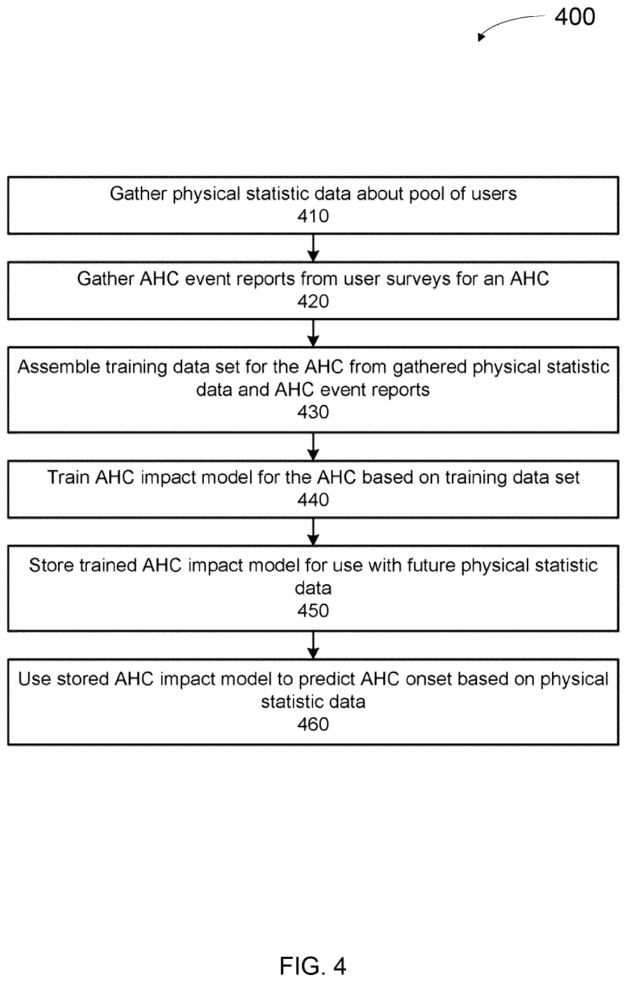

FIG. 4 is a flowchart illustrating an example process for generating a model of an acute health condition at a machine learning prediction system, according to an embodiment. The process 400 of FIG. 4 begins when a machine learning prediction system gathers 410 physical statistic data about a pool of users. For example, as described above, the machine learning prediction system can gather information from one or more health sensors of users of the machine learning prediction system. Then, the machine learning prediction system can gather 420 AHC event reports describing symptoms and/or confirmed cases of the AHC among the pool of users. For example, the gathered from user surveys as described above. Next, the machine learning prediction system can assemble 430 a training data set for the AHC using the gathered physical statistic data and AHC event reports and train 440 an AHC impact model for the AHC based on the assembled training data set. The trained AHC impact model can then be stored 450 and used 460 for prediction of AHC onset in users based on current physical statistic data. Similarly, an AHC impact model can be used to estimate the burden of the AHC on a population.

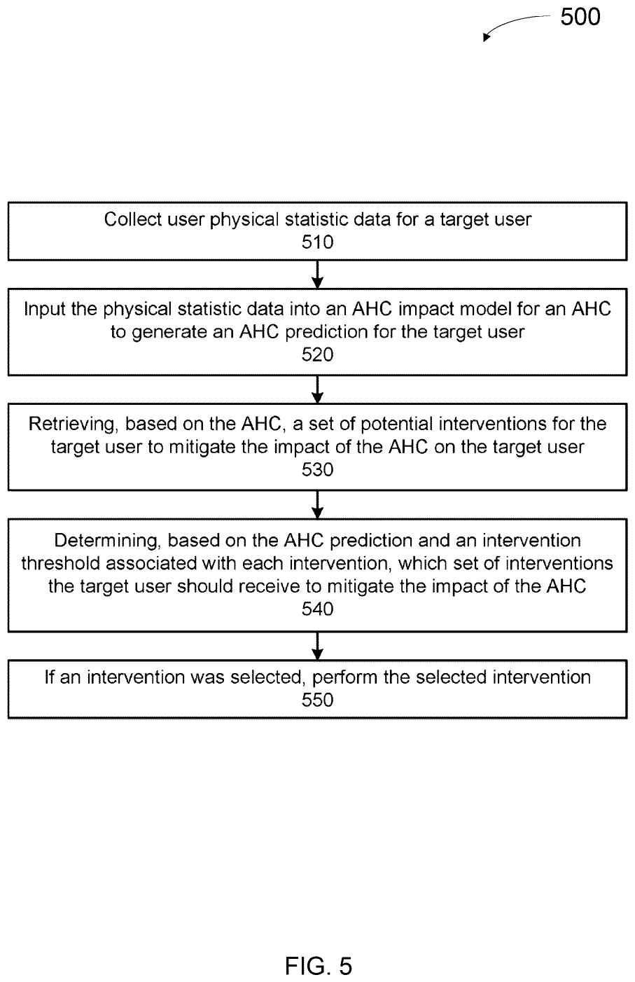

FIG. 5 is a flowchart illustrating an example process for providing an intervention to an individual user based on predicted onset of an acute health condition for the individual user based on a model of an acute health condition, according to an embodiment. The process 500 of FIG. 5 begins when a machine learning prediction system collects 510 user physical statistic data for a target user and inputs 520 the physical statistic data into an AHC impact model for an AHC. As described above, an AHC impact model can generate an AHC prediction for the target user including a confidence level or probability that the target user currently has the AHC and/or will soon experience the onset of symptoms of the AHC based on the target user's physical statistic data. Then, the machine learning prediction system can then retrieve 530 a set of potential interventions for the AHC which if implemented by the machine learning prediction system and/or the target user could mitigate the impact of the AHC on the user (or the population in the case of social distancing or other similar spread-limiting interventions). Out of the set of the interventions the machine learning prediction system determines 540 which interventions the target user should receive based on an intervention threshold associated with each intervention and the AHC prediction generated by the AHC impact model. If an intervention was selected, the machine learning prediction system can perform 550 the selected interventions, in some cases with the help of a third-party intervention system, as described above.

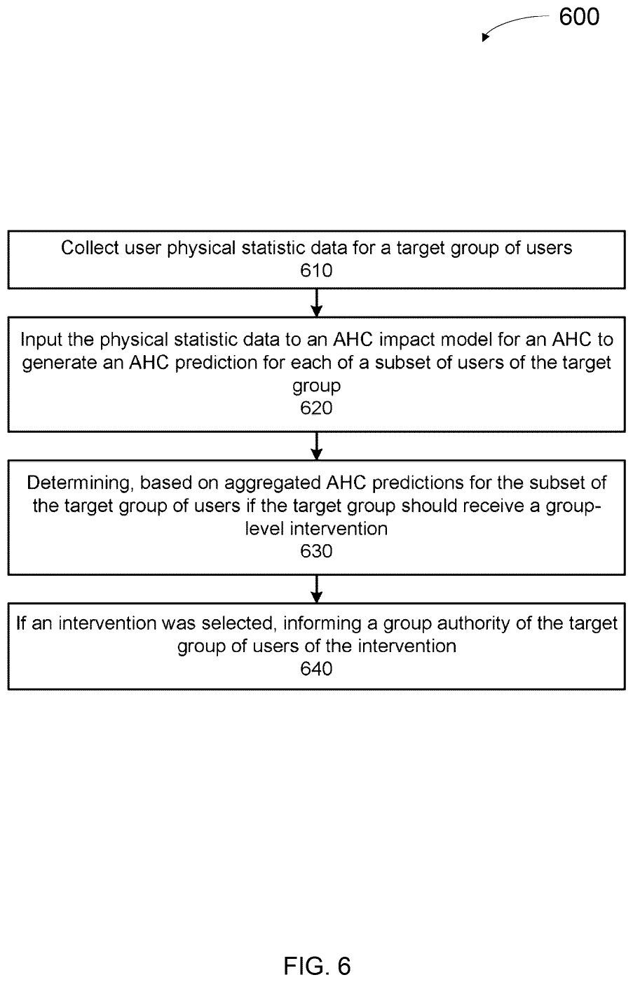

FIG. 6 is a flowchart illustrating an example process for determining a group level intervention for a target group using a model of an acute health condition to predict AHC onset for individual users of the target group, according to an embodiment. The process 600 of FIG. 6 begins when a machine learning prediction system collects 610 user physical statistic data for users of a target group of users. Then, the machine learning prediction system inputs 620 gathered physical statistic data of a subset of users of the target group into an AHC impact model for an AHC. As described above, an AHC impact model can generate an AHC prediction for an individual user, for example including a confidence level or probability that the target user currently has the AHC and/or will soon experience the onset of symptoms of the AHC based on the target user's physical statistic data. Using the set of individual AHC predictions for each user of the subset of users, the machine learning prediction system can determine 630 if the target group should receive a group-level intervention (such as cancellation of an event or a policy change). The machine learning prediction system can then inform 640 a group authority of the target group of one or more recommended group-level interventions.

### Recovery Trajectory Overview

In some cases, the disclosed method uses machine learning to better understand how particular patients may respond to surgical or medical procedures, or acute debilitating events. Using data collected by wearable sensors from patients with different physical characteristics and personal attributes, the disclosed method may predict a patient's time to recovery. The system may use a machine learning model trained on patient wearable device sensor data collected prior to and following an event. Based at least in part on analysis of this wearable device sensor data, which may include, but is not limited to, step count, heart rate, and sleep efficiency, the system may make a prediction as to at which point a patient will be fully recovered (i.e., a recovery time or time to recovery).

### Data Collection and Sensors

A wearable device may comprise one or more sensors to measure physical attributes of a human subject. For example, the wearable device may include one or more accelerometers, heart rate sensors, barometers, orientation sensors, or gyroscopes. The wearable device may include one or more cameras (e.g., red-green-blue (RGB), YUV, or depth), radar sensors, microphones, infrared sensors, or sensors configured to measure electromagnetic signals (e.g., electrodes or magnetometers). The sensors may be implantable, physically coupled to the body, or not contacted with the body.

The sensors of the wearable device may be configured to measure one or more quantities indicative of a subject's physical health or biophysical characteristics. For example, the sensors may be configured to measure step count, heart rate, sleep efficiency (the total number of minutes slept divided by the overall time in the bed), sleep quality, disordered sleep, respiration, blood oxygen, blood pressure, pulse rate, body temperature, gaze direction, glucose, or another health-related quantity. In some embodiments, the system may analyze data from at least one of sleep efficiency, step count, and heart rate data. In some embodiments, the system may analyze data from at least two of sleep efficiency, step count, and heart rate data.

The sensors may collect subject health data from before and after a debilitating event. The debilitating event may be a health intervention. The health intervention may be surgery. The surgery may be any surgery that causes a major and short-term disruption in mobility, sleep, or physiology. The surgery may be lower limb surgery. The surgery may be weight loss surgery. In some embodiments, the methods and systems disclosed herein may be configured to predict times to recovery from debilitating events that do not comprise weight loss surgery. The lower limb surgery may be bone repair surgery, ligament surgery, tendon surgery, knee or knee replacement surgery, or hip replacement surgery. The surgery may be open heart surgery, spine or neurosurgery, surgery involving lungs or otherwise the respiratory apparatus.

The recovery time may be from an illness, such as COVID, flu, or another acute condition for which the onset date is known with accuracy. The recovery time may be from a trauma, the trauma may be an injury, the injury may be an ankle sprain, Achilles rupture, or other ligament tear.

Health data may be collected for a first time period before the acute or debilitating event (or “event”) occurs. The health data may be collected at least one week, at least two weeks, at least three weeks, at least four weeks, at least five weeks, at least six weeks, at least seven weeks, at least eight weeks, at least nine weeks, at least ten weeks, at least 15 weeks, at least 20 weeks, at least 25 weeks, or at least 30 weeks before the health procedure. The health data may be collected at most one week, at most two weeks, at most three weeks, at most four weeks, at most five weeks, at most six weeks, at most seven weeks, at most eight weeks, at most nine weeks, at most ten weeks, at most 15 weeks, at most 20 weeks, at most 25 weeks, or at most 30 weeks before the acute or debilitating event. The health data may be collected between one and two weeks, between two and three weeks, between three and five weeks, between five and ten weeks, between ten and fifteen weeks, between 15 and 20 weeks, or between 20 and 30 weeks before the acute or debilitating event.

Health data may be collected for a second time period after the acute or debilitating event (or “event”) occurs. The health data may be collected at least one week, at least two weeks, at least three weeks, at least four weeks, at least five weeks, at least six weeks, at least seven weeks, at least eight weeks, at least nine weeks, at least ten weeks, at least 15 weeks, at least 20 weeks, at least 25 weeks, or at least 30 weeks after the acute or debilitating event. The health data may be collected at most one week, at most two weeks, at most three weeks, at most four weeks, at most five weeks, at most six weeks, at most seven weeks, at most eight weeks, at most nine weeks, at most ten weeks, at most 15 weeks, at most 20 weeks, at most 25 weeks, or at most 30 weeks after the acute or debilitating event. The health data may be collected between one and two weeks, between two and three weeks, between three and five weeks, between five and ten weeks, between ten and fifteen weeks, between 15 and 20 weeks, or between 20 and 30 weeks after the event.

The health data may be collected at a high frequency. For example, the health data may be collected at least once every minute, at least once every ten minutes, at least once every 15 minutes, at least once every 30 minutes, at least once every hour, at least once every two hours, at least once every three hours, at least once every six hours, at least once every 12 hours, at least once every day, or at least once every week. The health data may be collected at most once every six hours, at most once every 12 hours, at most once every day, or at most once every week.

### Other Data

The disclosed machine learning system may use non-wearable data in addition to wearable sensor data when making predictions. For example, the system may use demographic or personal data about a human subject. The data may include age, weight, height, fitness level or exercise frequency, types of exercise performed, gender, sex, location, medical history, family medical history, medications taken, wearable device usage patterns, occupation, or other data.

### Subject

The subject may be a human subject. The subject may be an animal subject. The subject may be a mammalian subject, such as a monkey, ape, mouse, rat, rabbit, dog, cat, pig, sheep, or cow. The subject may be a bird, such as a chicken, duck, or pigeon. The subject may be a reptile, such as a snake, lizard, or crocodilian. The methods disclosed herein may apply to debilitating events faced by animals, such as avian influenza.

In some embodiments, data from the subject, such as time to recovery, may be reported by the subject. In some embodiments, such data may be reported by a health care provider or another third party. In some embodiments, such data may be reported by an automated system.

### Machine Learning Algorithms

The disclosed system may use one or more machine learning algorithms to predict recovery time from sensor data. For example, the disclosed system may use a support vector machine, a logistic regression (e.g., using LASSO), a decision tree method (e.g., gradient boosted trees or random forest), or a neural network (e.g., a recurrent neural network). The system may use deep learning (e.g., a deep neural network).

### System and Method

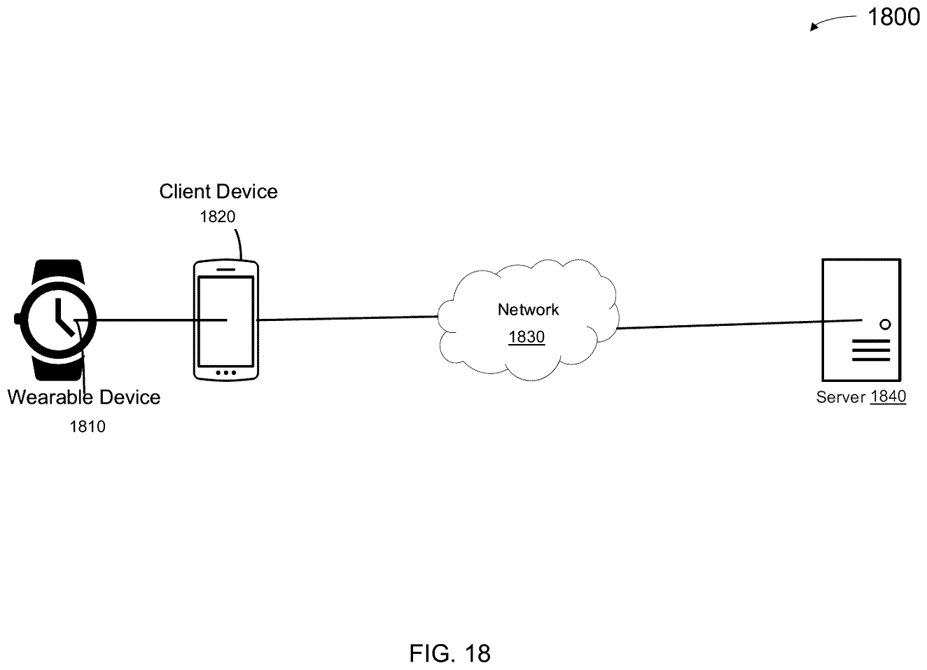

FIG. 18 illustrates a system 1800 for predicting a time to recovery (or recovery time) for a subject. The system may include one or more wearable devices 1810, a client device 1820, a network 1830, and a server 1840.

The wearable device 1810 and the client device 1820 may be coextensive or may be separate devices. In general, the wearable device 1810 may comprise one or more wearable device sensors (also referred to herein as “sensors”) for collecting patient health data and may include a capability to connect to a network (e.g., the network 1830) to transfer the sensor data to other components of the system 1800. The wearable device 1810 may be a watch, headgear, jewelry, clothing, fabric, footwear, headband, eyewear, or other article or electronic device configured to contact the skin of or a body part of the subject, and which may include or may be communicatively coupled to electronic circuitry that may collect, transmit, and/or process electrical signals derived from the subject. For example, the wearable device 1810 may be a Fitbit® or APPLE® Watch. The wearable device may comprise a sleep sensor to measure sleep efficiency, a heart rate sensor to measure heart rate, and/or a step count sensor (e.g., a pedometer) to measure step count.

The client device 1820 may be a computing device configured to access an application enabling a subject to self-report data. The client device 1820 may be a mobile computing device. The client device may be a smartphone, wearable device, cell phone, personal digital assistant (PDA), tablet computer, laptop computer, desktop computer, or other computing device. The application may be installed natively on the client device or may be accessible via a browsing application. The application may enable a subject to self-report recovery from surgery. The application may also enable a subject to track a progression or recovery trajectory from an acute or debilitating event (e.g., a surgery).

The server 1840 may maintain user or subject data and perform analysis of the data. For example, the server may store one or more machine learning models used to perform analysis of wearable data received from the subject as well as, optionally, subject-reported demographic or personal data. The server 1840 may use the machine learning models to make one or more predictions about a time to recovery for one or more users. The server 1840 may be a physical or cloud server. A physical server may comprise one or more computing devices.

The network 1830 may be a hardware and software system configured to enable the computing components of the system 1800 to communicate electronically and share resources with one another. The network 1830 may be the Internet, a local area network (LAN), or a wide area network (WAN).

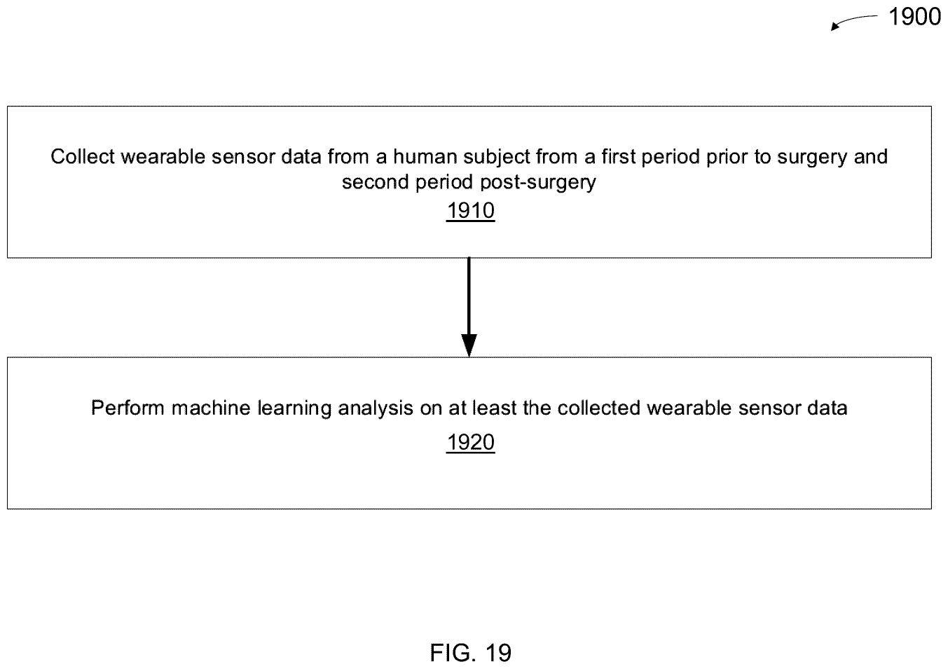

FIG. 19 illustrates a process 1900 for predicting recovery time (or “time to recovery”) from a debilitating or acute event.

In a first operation 1910, the system may collect wearable sensor data from a human subject for a first period prior to an acute or debilitating event and for a second period after the acute or debilitating event. In some embodiments, the first period is shorter than the second period. In some embodiments, the first period is the same length as the second period. In some embodiments, the first period is longer than the second period. The first period may be, for example, 12 weeks prior to surgery. The second period may be, for example, 26 weeks following surgery. The wearable sensor data may be collected daily. The wearable sensor data may comprise subject health measurements. For example, the wearable sensor data may comprise heart rate, step count, and sleep efficiency.

In a second operation 1920, the system may perform machine learning analysis on at least the collected wearable sensor data. The machine learning analysis may comprise a decision tree-based model (e.g., XGBoost). The machine learning analysis may generate a prediction for a post-event recovery time.

The prediction may be a binary prediction. For example, the system may predict a fast recovery time or a slow recovery time. A fast recovery time may be, for example, two months or less. A slow recovery time may be, for example, three months or more.

The prediction may be a multiclass prediction. For example, the system may predict a recovery time which may fall into one of the following categories: zero to one month, one to two months, two to three months, three to four months, or more than four months.

### Recovery Score

In some embodiments, the system may compute a personalized, real-time recovery score for a subject during the recovery period.

For a large database of recovery person-generated health data (PGHD) for a population, the system may, for a particular subject or individual, select a group of 20 similar individuals (“similarity group”) from the population. The similarity of the group may be based on individual characteristics, such as age, gender, type of acute or debilitating event suffered, time elapsed since diagnosis, another statistic, or a combination thereof. The similarity may be assessed using a distance function such as Euclidean, Mahalanobis, cosine similarity, or another function, between the vector representing the characteristic of the individual and the vector representing the same characteristics for other individuals whose similarity is being evaluated. For a particular health statistic or quantity of interest (e.g., step count, heart rate, or sleep efficiency), the system may compute a distribution for the similarity group and rank the subject within the group. For example, within the group, the system may calculate a percentile ranking for step count. The system may also average these rankings to produce an overall score in real-time. The system may also use the probability of full recovery within (e.g., six months) as computed by the machine learning system and may calculate a percentile ranking of that probability within the group. The score may be updated as the system receives additional data (e.g., self-reported or generated by wearable device) from the user.

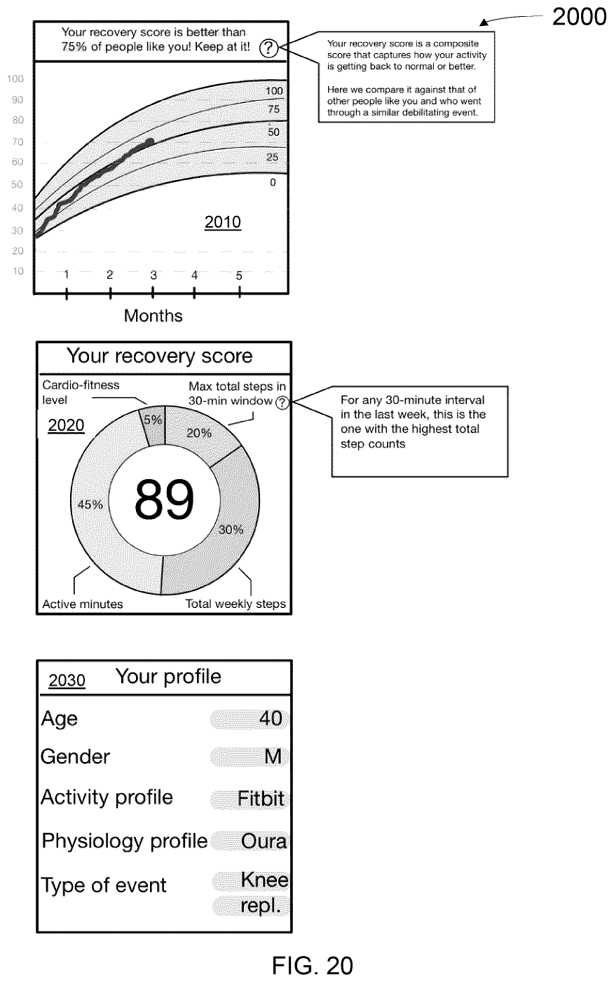

FIG. 20 illustrates screen captures 2010, 2020, 2030 of a user interface providing a score to a subject. The user interface may belong to a mobile device application. In a first screen capture 2010, a user's percentile score over time is overlaid on scores from users in the subject's similarity group. In this particular case, a score may representing probability of full recovery within six months rescaled over the range 0-100, as predicted by the machine learning system based on wearables and self-reported data available at the day the score is computed. The interface may inform the subject that recovery has progressed better than recovery for 75% of users in the similarity group, meaning that their probability of recovery at six months is higher than those of 75% of individuals in a similarity group. In a second screen capture 2020, the user interface displays the components of the subject's recovery score. This score may represent the probability of recovery at six months based on data available at the time the score is produced (e.g., at month three, as represented in the figure). This value may be generated as a prediction by the machine learning system. In this example, the contributions are 5% from cardio-fitness level, 20% maximum steps in a 30-minute window, 30% total weekly steps, and 45% active minutes.

### Training Phase

A machine learning software module may be provided by a server (e.g., the server 1240) and may implement one or more machine learning algorithms. A machine learning software module as described herein is configured to undergo at least one training phase wherein the machine learning software module is trained to carry out one or more tasks including data extraction, data analysis, and generation of output.

In some embodiments of the software application described herein, the software application comprises a training module that trains the machine learning software module. The training module is configured to provide training data to the machine learning software module, the training data comprising, for example, wearable sensor data, the date (e.g., precise to the day), of occurrence of an acute or debilitating event, and ground truth data comprising self-reported times to recovery (or recovery times), once recovery is completed or can no longer be attained (no recovery). In additional embodiments, said training data is comprised of wearable sensor dataand recovery times with corresponding subject personal and/or demographic data. In some embodiments of a machine learning software module described herein, a machine learning software module utilizes automatic statistical analysis of data to determine which features to extract and/or analyze from wearable sensor data. In some of these embodiments, the machine learning software module determines which features to extract and/or analyze from subject health data based on the training that the machine learning software module receives.

In some embodiments, a machine learning software module is trained using a data set and a target in a manner that might be described as supervised learning. In these embodiments, the data set is conventionally divided into a training set, a test set, and, in some cases, a validation set. In some embodiments, the data set is divided into a training set and a validation set. A target is specified that contains the correct classification of each input value in the data set. For example, a set of wearable sensor data from one or more individuals is repeatedly presented to the machine learning software module, and for each sample presented during training, the output generated by the machine learning software module is compared with the desired target. The difference between the target and the set of input samples is calculated, and the machine learning software module is modified to cause the output to more closely approximate the desired target value. In some embodiments, a back-propagation algorithm is utilized to cause the output to more closely approximate the desired target value. After many training iterations, the machine learning software module output will closely match the desired target for each sample in the input training set. Subsequently, when new input data, not used during training, is presented to the machine learning software module, it may generate an output classification value indicating which of the categories the new sample is most likely to fall into. The machine learning software module is said to be able to “generalize” from its training to new, previously unseen input samples. This feature of a machine learning software module allows it to be used to classify almost any input data which has a mathematically formulatable relationship to the category to which it should be assigned.

In some embodiments of the machine learning software module described herein, the machine learning software module utilizes an individual learning model. An individual learning model is based on the machine learning software module having trained on data from a single individual and thus, a machine learning software module that utilizes an individual learning model is configured to be used on a single individual on whose data it trained, or on individuals deemed similar to the individual on whose data it trained. Similarity may be defined in terms of a distance function (e.g., Euclidean, Mahalanobis, cosine similarity) between vectors containing variables characterizing two individuals, such as demographics, social determinant of health. It may be defined as distance in the space where those vectors are embedded (e.g., using autoencoder embedding techniques).

In some embodiments of the machine training software module described herein, the machine training software module utilizes a global training model. A global training model is based on the machine training software module having trained on data from multiple individuals and thus, a machine training software module that utilizes a global training model is configured to be used on multiple patients/individuals.

In some embodiments of the machine training software module described herein, the machine training software module utilizes a simulated training model. A simulated training model is based on the machine training software module having trained on data from wearable sensor data. A machine training software module that utilizes a simulated training model is configured to be used on multiple patients/individuals.

In some embodiments, the use of training models changes as the availability of wearable sensor data changes. For instance, a simulated training model may be used if there are insufficient quantities of appropriate patient data available for training the machine training software module to a desired accuracy. As additional data becomes available, the training model can change to a global or individual model. In some embodiments, a mixture of training models may be used to train the machine training software module. For example, a simulated and global training model may be used, utilizing a mixture of multiple patients' data and simulated data to meet training data requirements.

Unsupervised learning is used, in some embodiments, to train a machine training software module to use input data such as, for example, wearable sensor data data and output, for example, a predicted recovery time. Unsupervised learning, in some embodiments, includes feature extraction which is performed by the machine learning software module on the input data. Extracted features may be used for visualization, for classification, for subsequent supervised training, and more generally for representing the input for subsequent storage or analysis. In some cases, each training case may consist of a plurality of wearable sensor data.

Machine learning software modules that are commonly used for unsupervised training include k-means clustering, mixtures of multinomial distributions, affinity propagation, discrete factor analysis, hidden Markov models, Boltzmann machines, restricted Boltzmann machines, autoencoders, convolutional autoencoders, recurrent neural network autoencoders, and long short-term memory autoencoders. While there are many unsupervised learning models, they all have in common that, for training, they require a training set consisting of biological sequences, without associated labels.

A machine learning software module may include a training phase and a prediction phase. The training phase is typically provided with data to train the machine learning algorithm. Non-limiting examples of types of data inputted into a machine learning software module for the purposes of training include medical image data, clinical data (e.g., from a health record), encoded data, encoded features, or metrics derived from wearable sensor data. Data that is inputted into the machine learning software module is used, in some embodiments, to construct a hypothesis function to determine a predicted recovery time. In some embodiments, a machine learning software module is configured to determine if the outcome of the hypothesis function was achieved and based on that analysis make a determination with respect to the data upon which the hypothesis function was constructed. That is, the outcome tends to either reinforce the hypothesis function with respect to the data upon which the hypothesis function was constructed or contradict the hypothesis function with respect to the data upon which the hypothesis function was constructed. In these embodiments, depending on how close the outcome tends to be to an outcome determined by the hypothesis function, the machine learning algorithm will either adopt, adjust, or abandon the hypothesis function with respect to the data upon which the hypothesis function was constructed. As such, the machine learning algorithm described herein dynamically learns through the training phase what characteristics of an input (e.g., data) are most predictive in determining whether the features of a patient's wearable data are associated with a particular time to recovery.

For example, a machine learning software module is provided with data on which to train so that it, for example, can determine the most salient features of a received wearable sensor data to operate on. The machine learning software modules described herein train as to how to analyze the wearable sensor data, rather than analyzing the wearable sensor data using pre-defined instructions. As such, the machine learning software modules described herein dynamically learn through training what characteristics of an input signal are most predictive in determining whether the features of wearable sensor data predict a particular time to recovery.

In some embodiments, training begins when the machine learning software module is given wearable sensor data and asked to determine a recovery time. The predicted time to recovery is then compared to the true time to recovery that corresponds to the wearable sensor data. An optimization technique such as gradient descent and backpropagation is used to update the weights in each layer of the machine learning software module to produce closer agreement between the time to recovery predicted by the machine learning software module, and the actual time to recovery. This process is repeated with new wearable sensor data and time to recovery data until the accuracy of the network has reached the desired level. An optimization technique is used to update the weights in each layer of the machine learning software module to produce closer agreement between the time to recovery predicted by the machine learning software module, and the true time to recovery. This process is repeated with new wearable sensor data and time to recovery data until the accuracy of the network has reached the desired level.

In some embodiments, an individual's time to recovery is inputted by the individual of the system (e.g., using a mobile device application). In some embodiments, an individual's time to recovery is inputted by an entity other than the individual. In some embodiments, the entity can be a healthcare provider, healthcare professional, family member or acquaintance. In additional embodiments, the entity can be the instantly described system, device or an additional system that analyzes wearable sensor data and provides data related to time to recovery.

In some embodiments, a strategy for the collection of training data is provided to ensure that the wearable sensor data represents a wide range of conditions to provide a broad training data set for the machine learning software module. For example, a prescribed number of measurements during a set period may be required as a section of a training data set. Additionally, these measurements can be prescribed as having a set amount of time between measurements. In some embodiments, wearable sensor data measurements taken with variations in a subject's physical state may be included in the training data set.

In general, a machine learning algorithm is trained using wearable sensor data and/or any features or metrics computed from the above said data with the corresponding ground-truth values. The training phase constructs a transformation function for predicting a time to recovery from wearable sensor data and/or any features or metrics computed from the above said data of the unknown patient. The machine learning algorithm dynamically learns through training what characteristics of input data are most predictive in determining a time to recovery. A prediction phase uses the constructed and optimized transformation function from the training phase to predict the time to recovery by using the wearable sensor data and/or any features or metrics computed from the above said data of the unknown patient.

### Prediction Phase

Following training, the machine learning algorithm is used to determine, for example, the time to recovery on which the system was trained using the prediction phase. With appropriate training data, the system can identify the time in the future at which a patient may be expected to recover.

The prediction phase uses the constructed and optimized hypothesis function from the training phase to predict a time to recovery from the wearable sensor data.

In some embodiments, a probability threshold can be used in conjunction with a final probability to determine whether or not the patient is expected to recover within a particular fixed time (e.g., six months). In some embodiments, the probability threshold is used to tune the sensitivity of the trained network. For example, the probability threshold can be 1%, 2%, 5%, 10%, 15%, 20%, 25%, 30%, 35%, 40%, 45%, 50%, 55%, 60%, 65%, 70%, 75%, 80%, 85%, 90%, 95%, 98% or 99%. In some embodiments, the probability threshold is adjusted if the accuracy, sensitivity or specificity falls below a predefined adjustment threshold. In some embodiments, the adjustment threshold is used to determine the parameters of the training period. For example, if the accuracy of the probability threshold falls below the adjustment threshold, the system can extend the training period and/or require additional wearable sensor data and/or times to recovery. In some embodiments, additional measurements and/or times to recovery can be included into the training data. In some embodiments, additional measurements and/or times to recovery can be used to refine the training data set.

### Gradient Boosting

Embodiments of this disclosure may be implemented using gradient boosting algorithms such as XGBoost, a version of the gradient boosting algorithm designed for efficacy, computational speed, and model performance.

Boosting may refer to a technique (e.g., an ensemble learning technique) for increasing performance (e.g., of a machine learning algorithm or model). In some embodiments, boosting may convert a weak hypothesis or weak learner (a learner may be a program used to learn a machine learning model from data) to a strong learner, increasing predictive accuracy of a machine learning model.

Boosting is an ensemble learning method. Ensemble learning is a process in which decisions from multiple machine learning (ML) models are combined to reduce errors and improve prediction when compared to a single ML model. Ensemble learning may use ensemble voting on aggregated decisions from multiple weak learners (which may use decision tree algorithms) to generate a strong prediction. A weak learner may be defined as a program that does not make accurate predictions or produces outputs that have weak correlations with actual or ground truth values. For XGboost or other gradient boosting algorithms, decision trees may form the bases for weak learners. A boosting algorithm may use sequential ensemble learning—i.e., it may create new weak learners and may sequentially combine their predictions to improve model performance. For a sequence of predictors, the boosting algorithm may fit a predictor to residual errors made by the previous predictor.

Predictors in a boosting algorithm may comprise decision trees. A decision tree may be a supervised machine learning algorithm used for predictive modeling of a dependent variable (target) based on input of several independent variables. Decision trees may be classification trees or regression trees. A classification tree may be a decision tree that identifies a class or category in which a fixed or categorical target variable would most likely fall. A regression tree may predict a value of a continuous variable.

Gradient boosting, in particular, is boosting that uses gradient descent to minimize errors. Gradient boosting may adjust weights during training iteratively using a gradient descent algorithm. This method may iteratively reduce the loss of a machine learning model. In this context, loss may be defined as a quantification of a negative consequence associated with a prediction error.

Gradient boosting algorithms may be regression algorithms or classification algorithms. A regression algorithm may use a mean-squared error (MSE) loss function, while a classification algorithm may use a logarithmic loss function.

Gradient boosting uses additive modeling, a process that adds a new decision tree at a time to a gradient boosting model to reduce the loss and therefore improve the predictive power of the model. The additive modeling process may combine the output of each new tree with the combined output of the preceding trees until the model loss is minimized below a threshold or a limit on the number of trees the model can use is reached. Each subsequent predictor that is added may be fit to the residual errors (i.e., the difference between the predicted value and the observed value) made by the previous predictor (assuming a MSE loss function).

Extreme gradient boosting (XGBoost) may enhance gradient boosting with advanced regularization (L1 and L2).

In some embodiments, the machine learning methods disclosed herein are implemented using other ensemble methods, other decision tree methods, or other boosting methods.

### Recovery Trajectory Experiment

The following sections describe setup and results of an experiment and should not be construed to limit this disclosure. Many of the procedures described with respect to this experiment may be used to determine predictions for other debilitating or acute events in addition to lower limb surgery events.

### Methods

Fitbit device data of steps, heart rate and sleep from 26 weeks before to 26 weeks after a self-reported surgery date was collected for 1,324 individuals who underwent surgery on a lower limb. Subgroups of individuals who self-reported surgeries for bone fracture repair (355 individuals), tendon or ligament repair/reconstruction (773), and knee or hip joint replacement (196) were identified. Linear mixed models were used to estimate average effect of time relative to surgery on daily activity measurements while adjusting for gender, age, and participant-specific activity baseline. For example, self-reported recovery time was predicted using XGBoost for a sub-cohort of 127 individuals with dense wearable data who underwent tendon or ligament surgery.

### RESULTS

The 1,324 study individuals were all U.S. residents, predominantly female (84%), white or Caucasian (85%) and young to middle-aged (mean 36.2 years). In some embodiments, 12-week pre- and 26-week post-surgery trajectories of daily behavioral measurements (step count, heart rate, sleep efficiency score) captured activity changes relative to an individual's baseline. Recovery trajectories differ across surgery types, recapitulate the documented effect of age on functional recovery, and highlight differences in relative activity change across self-reported recovery time groups. Finally, in the case of a sub-cohort of 127 individuals, long-term recovery can be accurately predicted, on an individual level, only 1 month after surgery (AUROC 0.734, AUPRC 0.8). In some embodiments predictions are most accurate when long-term, individual baseline data are available.

### Data Collection

The experiment used an online platform where people can connect their digital health tools, including wearable activity trackers and fitness apps. This platform enables rapid recruitment of participants to specific studies, where consent for all research is granted on a per use basis.

Data was collected from a previously cited study, surveying participant experience relating to surgery and medical devices. Briefly, participants were asked about which surgeries they had experienced, and for the most recent surgery, the type of surgery, the date of surgery and the time required for recovery. The full survey is included in Supplementary Note 1. Between May 5 and September 21 of 2018, 200,325 individuals consented to take part in the study. 50,938 participants reported they underwent a medical procedure, out of which 4,312reported at least one of the three lower limb procedures itemized in the survey (surgery to repair a bone fracture, tendon or ligament repair/reconstruction surgery, or knee or hip joint replacement surgery). The initial dataset consisted of 3,740 participants reporting lower limb surgery as their most recent surgery.

### Data Processing

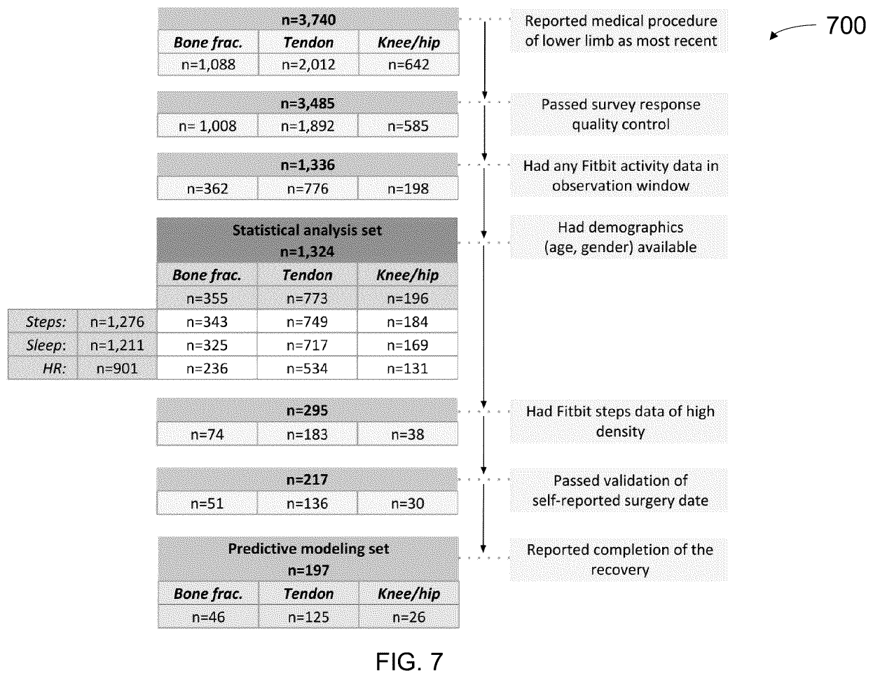

The participants' filtering process is illustrated in FIG. 7. From the initial dataset, participants who had multiple unique answers to questions about the most recent procedure type, or recovery time, or who provided an implausible recovery time label were filtered out (for example, reported recovery time of “3-5 months” where procedure date was less than 3 months from the survey date). The resulting data set consisted of 3,485 participants.

Next, with the participants' permission, their activity datasets were linked for the time window from 182 days (26 weeks) before to 182 days after the self-reported surgery date. To ensure consistency in data quality across the participants, only participants who had any Fitbit device data available in the observation window (n=1,336) were kept. Fitbit devices have been validated and reported as reliable for capturing steps, heart rate, and sleep data; these three data modalities were used to get daily aggregates of various activity and behavioral statistics (see details in Supplementary Note 2). All three modalities are known to be of relevance to post-surgical recovery.

Further, only participants for which age and gender data were available (n=1,324) were kept. Most of the participants had steps (n=1,276) and sleep (n=1,211) data available, fewer participants had heart rate data (n=901). At this point, no participant exclusion criterion due to missing data was applied; data missingness in statistical analysis part of this work is addressed by the choice of a modeling approach, as described below.

Prediction of recovery time required further data filtering to ensure higher data density on a participant-level so that a prediction could be made for each individual. This was achieved by restricting the data sets to participants who (a) did not have continuous periods of missing steps data longer than 28 days, and (b) had at least 50% of observation window days with steps data available. Data coverage in the full statistical analysis data sample (n=1,324) and filtered sample (n=295) is illustrated in Supplementary Note 3.

In order to ensure maximal data quality in the reporting of surgery dates, cases with high likelihood of mis-reporting were systematically identified using a change point detection methodology. This approach was adapted from; a function was fit based on the cohort-level model, and excluded instances where the function strongly fit, but the self-reported and function-reported surgery date were more than 28 days apart. The process is described in more detail in Supplementary Note 4. After applying the rule, n=217 out of 295 participants remained. Finally, only participants who reported completion of the recovery were kept. The final predictive modeling sample had n=197 participants.

### Statistical Modeling of Wearable Person-Generated Health Data (PGHD)

To estimate the impact of medical procedure on steps count, heart rate and sleep, the statistical analysis focused on three activity features: total number of steps, 95th percentile heart rate, and sleep efficiency (the proportion of minutes asleep of the total time in bed) during the main sleep. The baseline time period was defined as weeks from 26 weeks before (the earliest week in observation window) to 13 weeks before the surgery; the upper limit of 13 weeks before the surgery was chosen in order to account for potential cases of relatively long time from injury to surgery (average of 13 weeks of time from injury to surgery was reported in patients with chronic Achilles tendon rupture, where more than half of the cases had tendon rupture after failure of conservative treatment). In all visualizations in the manuscript, “week 0” label denotes a 7 days-long period starting on a self-reported surgery day. Daily activity measurements were modeled with a linear mixed effect model (LMM), fitting a separate model for each activity feature and surgery type subcohort. The outcome was defined as the participant- and day-specific activity measurement. The baseline period and each week in range from 12 before to 26 after the surgery were represented by an indicator variable. The model was adjusted for fixed effects of age, age and relative week interaction, gender, month of the year, weekend day vs. weekday, and participant-specific random effects (baseline activity and weekend day vs. weekday).

To further estimate trajectories of activity across time of recovery groups, the above model was extended by adding indicator variables for self-reported recovery time groups and for recovery group and relative week interaction.

The choice of using day-level activity measurements and employing LMM with participant-specific intercept can avoid a need to enforcing minimal data coverage or performing missing data imputation. Importantly, by including participants with data missingness, statistical power was increased and biasing in population-level estimates of activity was avoided.

### Prediction of Self-Reported Recovery Times

To demonstrate utility of wearable PGHD in predicting long-term trajectories of mobility recovery, the experiment was designed to evaluate performance of classifying self-reported recovery time labels. The machine learning task setup is described in more detail in Supplementary Note 5. In short, the model's performance was compared in six scenarios which differed in assumed availability of PGHD from wearable sensors: (1) no post-operative, no pre-operative, (2) no post-operative, 6 months (full) pre-operative, (3) 4 weeks post-operative, no pre-operative, (4) 4 weeks post-operative, 6 months pre-operative, (5) 6 months (full) post-operative, no pre-operative, (6) 6 months post-operative, 6 months pre-operative; in each (1)-(6) case, demographics (age, gender) information was used.

Due to relatively small sample sizes for bone fracture and knee/hip replacement surgery predictive data sets (n=46 and 26, respectively; see Table 2), the experiment was narrowed down to analyzing the tendon/ligament surgery group only (n=125), and the task was cast as a binary classification of a participant into a faster (“0-2 months”; coded as negative case) and slower (“>=3 months”; coded as positive case) track of mobility recovery. The classification models were trained with the Extreme Gradient Boosting (XGBoost) algorithm and evaluated in the 100-repeat holdout procedure. Alternative algorithms, including random forest with data imputation and feature preselection, and LASSO logistic regression were explored in preliminary stages of analysis and they did not yield performance results better than XGBoost (data not shown). Area under the ROC curve (AUROC) and area under the precision-recall curve (AUPRC) values, obtained on holdout test set across the 100 repetitions, are reported.

### Study Participants

Table 2 shows a summary of participants demographics and self-reported recovery time for statistical modeling sample (n=1,324) and predictive modeling sample (n=197). Data are summarized for whole sample cohorts (“All”) and for respective strata by surgery type. Participants included in the statistical analysis sample were predominantly female (84%), white or Caucasian (85%), college educated (62%), and young to middle-aged (mean [sd] 36.2 [12.9] years), closely in line with distribution skewness we observed for the whole user base of the Achievement platform (77% female, 88% white or Caucasian, mean age 33 years). The mean age varied across the surgery type sub-cohorts, from 32.9 in bone fracture surgery to 47.7 in knee/hip joint replacement surgery sub-cohort; for comparison, the average age for total hip arthroplasty and total knee arthroplasty patients were reported equal 65 years and 67 years, respectively. The most common self-reported time of recovery fell between 1 and 5 months for bone fracture and knee/hip replacement surgery, and from 1 to 12 months for tendon/ligament repair surgery.

Demographic data summaries for participants included in the predictive modeling sample follow closely the distribution of the analysis data sample. The percentages of self-reported time groups changed mostly due to the fact this sample excluded individuals who have not reported completion of the recovery.

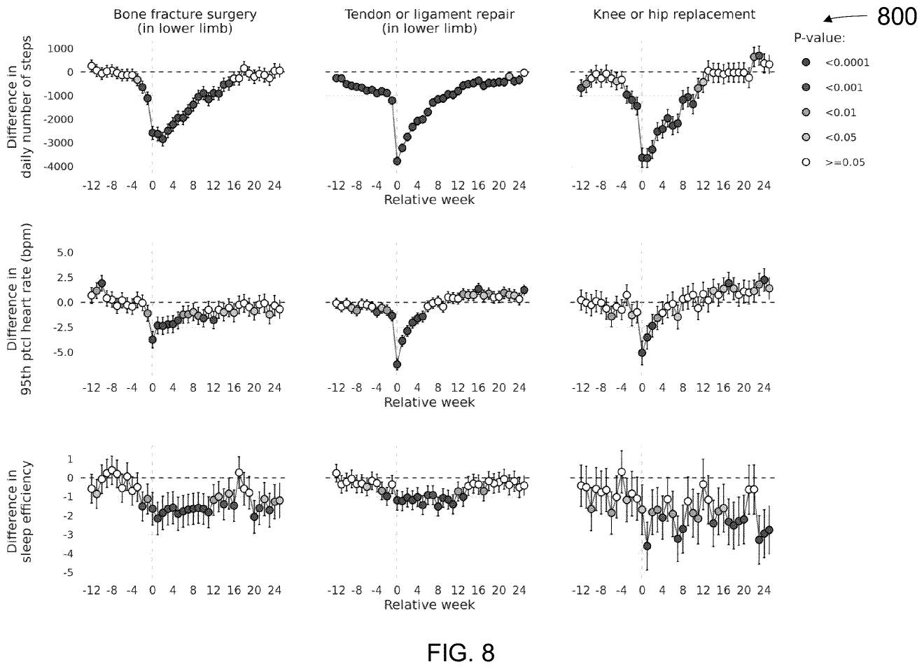

FIG. 8 summarizes the resulting cohort-level model fit, showing, for each surgery type, changes relative to baseline for representative features from step, heart rate and sleep data (daily step count, 95th percentile heart rate and sleep efficiency, respectively) for weeks from 12 before to 26 after the surgery. The trajectories are shown for a “typical” cohort individual (female at age 40, with average baseline activity level among otherwise similar ones). Model-estimated values of activity are also summarized in Table 4 in Supplementary Note 8.

At baseline, the estimated average daily measurement values varied very slightly across three surgery type subcohorts and equal: 8900, 8905, and 8815 for daily sum of steps, 103.9, 102.9, and 103.8 for 95th percentile of heart rate (bpm), 60.4, 57.6, and 57.7 for sleep efficiency-for three surgery type subcohorts (bone fracture repair, tendon or ligament repair/reconstruction, knee or hip joint replacement), respectively. As expected, all surgeries resulted in significant changes in activity, typically reducing daily step counts by 3000 to 4000 steps in the week following surgery, returning to near baseline levels over 8 to 12 weeks. All surgeries also resulted in reductions in submaximal heart rate which generally returned to baseline levels within 4 to 8 weeks and reductions in sleep efficiency which remained throughout the 12 weeks post-surgery. Activity and heart rate data were generally observed to be less variable than sleep data, possibly due to poorer nighttime data coverage and relatively low accuracy of current models for estimating sleep metrics from consumer wearables.

In addition to these general similarities, patterns were also observed that distinguished the three surgery groups and which correspond to distinct best practices. For example, significant pre-surgical reduction in steps sum and heart rate levels was seen in the 2 to 3 weeks prior to bone fracture surgeries, whereas for tendon and ligament surgeries this reduction was already apparent 8 to 10 weeks prior to surgery and for knee or hip replacement the reduction was stronger (more than 1000 steps) and observable 3 to 4 weeks prior to surgery. Distinct post-surgical recovery trajectories were also observed, for example, the effect of bed rest in bone fracture and joint replacement surgeries was visible immediately post surgery, while tendon/ligament repair surgery patients recovered to baseline activity more slowly than the two other groups, which agrees with a slightly higher proportion of self-reported “6-12 months” time of recovery for this group (see Table 2). To confirm the validity of the model, the known effect of age on recovery trajectories was captured (see Supplementary Note 6).

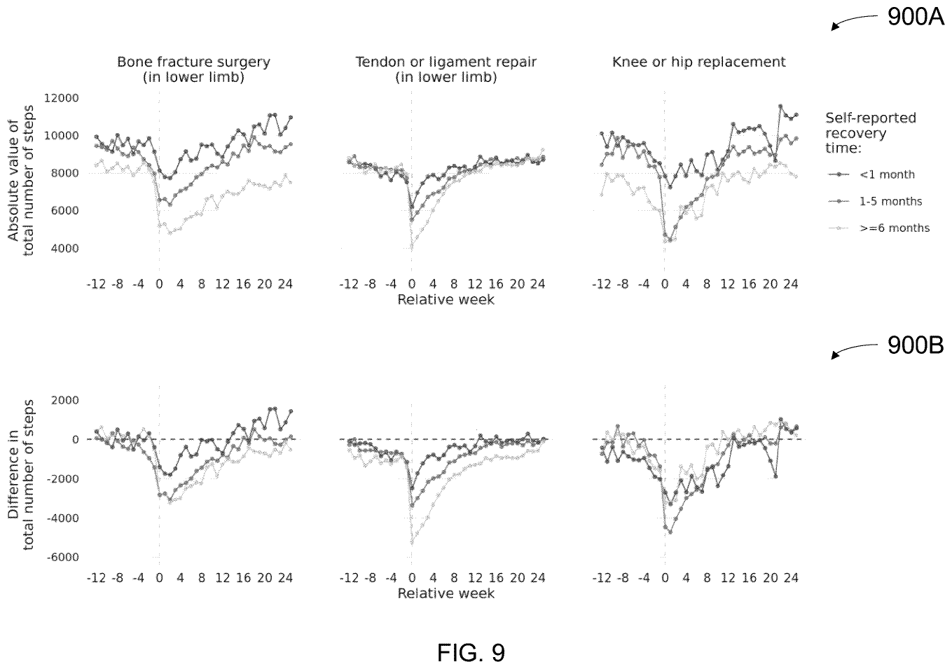

To verify that PGHD from wearable sensors can capture differences in activity across recovery groups, an extended statistical model (see: Methods) was used. FIG. 9 shows estimated average trajectories of daily number of steps across three self-reported recovery time groups, across the three lower limb surgeries. Values are shown for a “typical” cohort individual (female at age 40, with average baseline activity level among similar ones). The upper panel shows absolute activity (steps) values, the bottom plots panel shows change with respect to the model-estimated baseline. In the 1-4 weeks post-operative period, absolute values of activity distinguish the recovery groups, especially for bone fracture and tendon/ligament repair groups. In some embodiments, there is a complementary signal in the trajectory of relative change compared to the baseline, particularly for the tendon/ligament repair subcohort, where differences between the recovery time groups were visible both before and after the surgery. For the knee/hip replacement surgery sub-cohort (the smallest subcohort), relatively higher variability of fitted values was observed; the resulting patterns may have possibly represented a mixture of different knee and hip replacement procedures' effects which cannot be disentangled based on the survey conducted.

Model-estimated values of activity are also summarized in Table 5 in Supplementary Note 8. For completion, activity trajectories estimated across two and across four self-reported recovery time groups are included in Supplementary Note 7.

### Wearable PGHD can be Used to Predict Recovery Trajectories

Table 3 summarizes the results of the experiment to discriminate participants who self-reported faster (“0-2 months”) versus slower (“>=3 months”) functional recovery trajectory, across six scenarios in which different data availability was assumed: demographic data only; individual baseline data only; 1-month post-surgery with and without an individual baseline; 6 months post-surgery with and without individual baseline. The analysis focused on the tendon or ligament surgery group (n=125) as the bone fracture (n=46) and knee/hip replacement (n=26) groups were too small to robustly train and test a predictive model.

Demographic variables (age, gender) themselves were not discriminative between faster and slower recovery track patients, attaining median AUROC of 0.489 (mean 0.473, standard deviation (sd) 0.108; see Table 3). This aligns to high demographic similarity between the recovery groups, for example in the tendon/ligament surgery group, the sample mean of age was very similar in the faster and slower recovery tracks, 36.6 (sd=10.9) and 35.9 (sd=11.3), respectively.

In the 4 weeks post-operative scenarios, the scenario with pre-operative activity data available attained higher AUROC (median=0.734, mean=0.724, sd=0.095) than in the scenario without pre-operative data (AUROC median=0.701, mean=0.705, sd=0.089).

Compared to 4 weeks post-operative scenarios, the 6 months post-operative scenarios yielded results slightly worse when pre-operative activity data were available (median=0.721, mean=0.71, sd=0.096) and slightly better without pre-operative activity data (median=0.716, mean=0.712, sd=0.084).

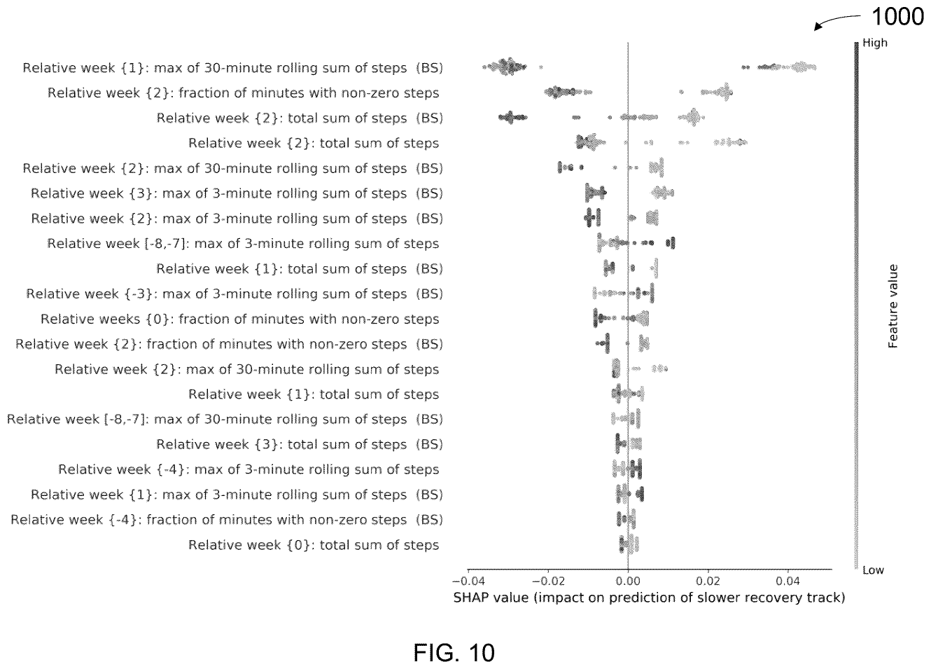

The features relative to baseline and those calculated from weeks immediately around the surgery were observed to be particularly important in driving the predictive power (see FIG. 10). Taken together, these results suggest that 4 weeks post-operative activity data already carry substantial information predictive of a patient's long-term recovery, and that the discriminative power of a model using 4 weeks post-operative activity data may be improved when pre-operative data were available.

### DISCUSSION/CONCLUSION

Functional recovery trajectories can be accurately modeled based on data from consumer wearable devices describing everyday function from up to 6 months prior to surgery to 6 months post-surgery. Similarly, typical recovery trajectories from different types of surgery can be distinguished, for example the 2-4 weeks of immobilization following bone fracture surgery, versus immediate remobilization of patients following tendon surgery. This model was supported using the known impact of age on functional recovery. Additionally, retrospective, recovery trajectories are clearly differentiated in terms of recovery trajectories, for example by the “depth” of functional limitation immediately post-surgery. Groups can additionally be differentiated based on pre-surgery, long-term baseline function and functional decreases immediately prior to surgery.

Prediction of long-term outcomes is highly important because early intervention, for example increasing exercise, is hypothesized to improve recovery outcomes. Indeed, higher levels of activity prior to surgery can correspond with better functional recovery post-surgery. The accurate prediction of outcomes is often not possible, as pre-surgery risk factors and demographics, without any functional baseline data, do not provide sufficient predictive power, for example 2-year risk of knee replacement revision. Passively collected, consumer-grade wearable data can provide baseline data to accurately predict long-term recovery trajectories. Furthermore, such predictions can be made only 1 month after surgery, early enough to inform alterations to physiotherapy regimes, for example specific targeting of “prehabilitation.” Recent work has also shown that this approach may have value in other therapeutic interventions, for example in oncology.

The data used to train the machine learning model is primarily based on self-reported dates and recovery times. In other embodiments, data to train the machine learning model may be extracted automatically by other sources, including electronic health records (HER), claims data, and from other sources, upon consent of the individual. Data used is conservatively collected to ensure maximal quality, in part enabled by the large scale of data collection. In other embodiments, data can be collected and used from a wider range of consumer devices.

In some implementations, adding more specific information about causes for surgical intervention may prevent further clustering or data analysis without.

### Figure Legends

FIG. 7 illustrates study participants' filtering process. Flow chart demonstrates number of participants across three lower limb surgery types: surgery to repair a bone fracture (“Bone frac.”), tendon or ligament repair/reconstruction surgery (“Tendon”), or knee or hip joint replacement surgery (“Knee/hip”).

FIG. 8 illustrates changes in activity features in subsequent weeks from week 12 before to week 26 after the surgery compared to average value in the baseline period (from week 26 to week 13 before the surgery). Horizontal plot panels correspond to three daily features: total number of steps, 95th percentile heart rate, and sleep efficiency during the main sleep. Vertical plot panels correspond to three lower limb surgery types: bone fracture, tendon or ligament repair, and knee or hip replacement. The colors and error bars correspond to p-value value bin and 95% confidence interval of model coefficient estimate for an effect of a relative week compared to baseline, respectively. The “week 0” label (x-axis) denotes a 7 days-long period starting on a self-reported surgery day.

FIG. 9 illustrates plots that show estimated trajectories of daily number of steps of subjects across three self-reported recovery time groups in subsequent weeks from 12 weeks before to 26 weeks after the surgery. The upper plots show absolute values of activity, the bottom plots show activity with respect to the model-estimated baseline. Vertical plot panels correspond to three lower limb surgery types: bone fracture, tendon or ligament repair, and knee or hip replacement. The color of a point/line corresponds to the self-reported recovery time group. The “week 0” label (x-axis) denotes a 7 day-long period starting on a self-reported surgery day.

FIG. 10 illustrates SHapley Additive explanations (SHAP) obtained from hand-tuned XGBoost model fitted to data of all participants in the tendon/ligament surgery group, assuming 4 weeks post-operative and 6 months pre-operative availability of PGHD from wearable sensors. The SHAP values are shown for the top 20 most impactful predictors. The suffix “(BS)” denotes predictors defined as a ratio of value derived from a particular week(s) period to value derived from the baseline period.

TABLE 2
Participants' demographics and self-reported recovery time for statistical modeling sample and predictive modeling sample. Data are
summarized for the whole sample cohort (“All”) and by strata by lower limb surgery types: surgery to repair a bone fracture (“Bone
frac.”), tendon or ligament repair/reconstruction surgery (“Tendon”), or knee or hip joint replacement surgery (“Knee/hip”).
Age at the time of procedure was estimated based on information from a patient ID-linked survey at a different time point than the medical event survey.

| | Statistical modeling set | | | | | | | | Predictive modeling set | | | | | | | |
|---|---|---|---|---|---|---|---|---|---|---|---|---|---|---|---|---|
| | All | | Bone frac. | | Tendon | | Knee/hip | | All | | Bone frac. | | Tendon | | Knee/hip | |
| | n = 1,324 | | n = 355 | | n = 773 | | n = 196 | | n = 197 | | n = 46 | | n = 125 | | n = 26 | |
| Gender | | | | | | | | | | | | | | | | |
| Female | 1,117 | (84%) | 307 | (86%) | 648 | (84%) | 162 | (83%) | 169 | (86%) | 41 | (89%) | 106 | (85%) | 22 | (85%) |
| Male | 203 | (15%) | 47 | (13%) | 122 | (16%) | 34 | (17%) | 28 | (14%) | 5 | (11%) | 19 | (15%) | 4 | (15%) |
| Other | 4 | (<1%) | 1 | (<1%) | 3 | (<1%) | 0 | (0%) | 0 | (0%) | 0 | (0%) | 0 | (0%) | 0 | (0%) |
| Race | | | | | | | | | | | | | | | | |
| White or | 1,119 | (85%) | 302 | (85%) | 649 | (84%) | 168 | (86%) | 168 | (85%) | 37 | (80%) | 110 | (88%) | 21 | (81%) |
| Caucasian | | | | | | | | | | | | | | | | |
| Black or | 52 | (4%) | 15 | (4%) | 30 | (4%) | 7 | (4%) | 5 | (3%) | 1 | (2%) | 4 | (3%) | 0 | (0%) |
| African | | | | | | | | | | | | | | | | |
| American | | | | | | | | | | | | | | | | |
| Hispanic or | 61 | (5%) | 14 | (4%) | 39 | (5%) | 8 | (4%) | 8 | (4%) | 2 | (4%) | 4 | (3%) | 2 | (8%) |
| Latino | | | | | | | | | | | | | | | | |
| Other | 45 | (3%) | 10 | (3%) | 26 | (3%) | 9 | (5%) | 6 | (3%) | 3 | (7%) | 2 | (2%) | 1 | (4%) |
| Unavailable | 47 | (4%) | 14 | (4%) | 29 | (4%) | 4 | (2%) | 10 | (5%) | 3 | (7%) | 5 | (4%) | 2 | (8%) |
| Age | | | | | | | | | | | | | | | | |
| mean (sd) | 36.2 | (12.9) | 32.9 | (11.1) | 34.9 | (11.7) | 47.7 | (14.4) | 37.3 | (11.6) | 34.2 | (8.9) | 36.1 | (11.1) | 48.4 | (12.4) |
| median [min, | 34 | [18, 77] | 31 | [18, 70] | 33 | [18, 70] | 51 | [18, 77] | 35 | [18, 71] | 32 | [19, 53] | 34 | [18, 64] | 49 | [24, 71] |
| max] | | | | | | | | | | | | | | | | |
| Education | | | | | | | | | | | | | | | | |
| Doctorate, MD | 29 | (2%) | 5 | (1%) | 19 | (2%) | 5 | (3%) | 4 | (2%) | 0 | (0%) | 4 | (3%) | 0 | (0%) |
| Graduate | 236 | (18%) | 58 | (16%) | 146 | (19%) | 32 | (16%) | 33 | (17%) | 8 | (17%) | 22 | (18%) | 3 | (12%) |
| degree | | | | | | | | | | | | | | | | |
| College degree | 515 | (39%) | 133 | (37%) | 317 | (41%) | 65 | (33%) | 89 | (45%) | 24 | (52%) | 58 | (46%) | 7 | (27%) |
| (AS or BS) | | | | | | | | | | | | | | | | |
| Some college | 311 | (23%) | 87 | (25%) | 183 | (24%) | 41 | (21%) | 42 | (21%) | 7 | (15%) | 26 | (21%) | 9 | (35%) |
| Trade or | 77 | (6%) | 23 | (6%) | 33 | (4%) | 21 | (11%) | 8 | (4%) | 4 | (9%) | 3 | (2%) | 1 | (4%) |
| vocational | | | | | | | | | | | | | | | | |
| training | | | | | | | | | | | | | | | | |
| High school | 107 | (8%) | 31 | (9%) | 48 | (6%) | 28 | (14%) | 13 | (7%) | 1 | (2%) | 8 | (6%) | 4 | (15%) |
| diploma/GED | | | | | | | | | | | | | | | | |
| No high school | 9 | (1%) | 5 | (1%) | 4 | (1%) | 0 | (0%) | 1 | (1%) | 0 | (0%) | 1 | (1%) | 0 | (0%) |
| diploma | | | | | | | | | | | | | | | | |
| Unavailable | 40 | (3%) | 13 | (4%) | 23 | (3%) | 4 | (2%) | 7 | (4%) | 2 | (4%) | 3 | (2%) | 2 | (8%) |
| Recovery time | | | | | | | | | | | | | | | | |
| <1 month | 260 | (20%) | 61 | (17%) | 164 | (21%) | 35 | (18%) | 37 | (19%) | 6 | (13%) | 26 | (21%) | 5 | (19%) |
| 1-2 months | 257 | (19%) | 80 | (23%) | 137 | (18%) | 40 | (20%) | 51 | (26%) | 16 | (35%) | 29 | (23%) | 6 | (23%) |
| 3-5 months | 292 | (22%) | 96 | (27%) | 151 | (20%) | 45 | (23%) | 52 | (26%) | 15 | (33%) | 28 | (22%) | 9 | (35%) |
| 6-12 months | 209 | (16%) | 38 | (11%) | 142 | (18%) | 29 | (15%) | 53 | (27%) | 7 | (15%) | 41 | (33%) | 5 | (19%) |
| 1 year or | 28 | (2%) | 8 | (2%) | 17 | (2%) | 3 | (2%) | 4 | (2%) | 2 | (4%) | 1 | (1%) | 1 | (4%) |
| longer | | | | | | | | | | | | | | | | |
| I never fully | 33 | (2%) | 8 | (2%) | 20 | (3%) | 5 | (3%) | 0 | (0%) | 0 | (0%) | 0 | (0%) | 0 | (0%) |
| recovered | | | | | | | | | | | | | | | | |
| I'm still | 245 | (19%) | 64 | (18%) | 142 | (18%) | 39 | (20%) | 0 | (0%) | 0 | (0%) | 0 | (0%) | 0 | (0%) |
| recovering | | | | | | | | | | | | | | | | |

TABLE 3
Performance of predictive models in the task of discriminating participants
between a faster (“0-2 months”) and slower (“>=3 months”) track of mobility recovery. Results
are shown across six experiment scenarios in which different data availability was assumed (in
each case, age and gender demographic information was used), and across surgery types
considered.

| Experiment scenario | | | | |
|---|---|---|---|---|
| (post-/pre-operative | AUROC | | AUPRC | |
| wearable PGHD | | median | | median |
| availability) | mean (sd) | [min, max] | mean (sd) | [min, max] |
| | Tendon or ligament repair/reconstruction surgery | | | |
| (1) no post-op, no pre-op | 0.473 (0.108) | 0.489 | 0.569 (0.059) | 0.563 |
| | | [0.114, | | [0.428, |
| | | 0.669] | | 0.727] |
| (2) no post-op, 6m pre- | 0.497 (0.096) | 0.498 | 0.596 (0.068) | 0.596 |
| op | | [0.266, | | [0.453, |
| | | 0.734] | | 0.741] |
| (3) 4wk post-op, no pre- | 0.705 (0.089) | 0.701 | 0.784 (0.076) | 0.799 |
| op | | [0.510, | | [0.598, |
| | | 0.929] | | 0.947] |
| (4) 4wk post-op, 6m pre- | 0.724 (0.095) | 0.734 | 0.795 (0.077) | 0.800 |
| op | | [0.442, | | [0.576, |
| | | 0.942] | | 0.960] |
| (5) 6m post-op, no pre- | 0.712 (0.084) | 0.716 | 0.798 (0.067) | 0.806 |
| op | | [0.542, | | [0.628, |
| | | 0.929] | | 0.937] |
| (6) 6m post-op, 6m pre- | 0.710 (0.096) | 0.721 | 0.786 (0.080) | 0.791 |
| op | | [0.435, | | [0.542, |
| | | 0.942] | | 0.960] |

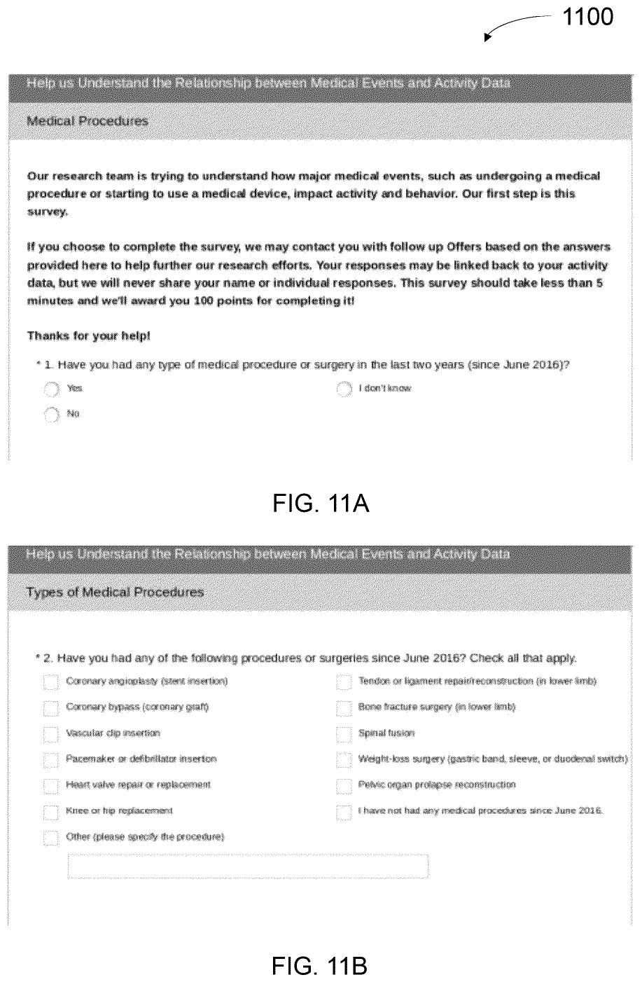

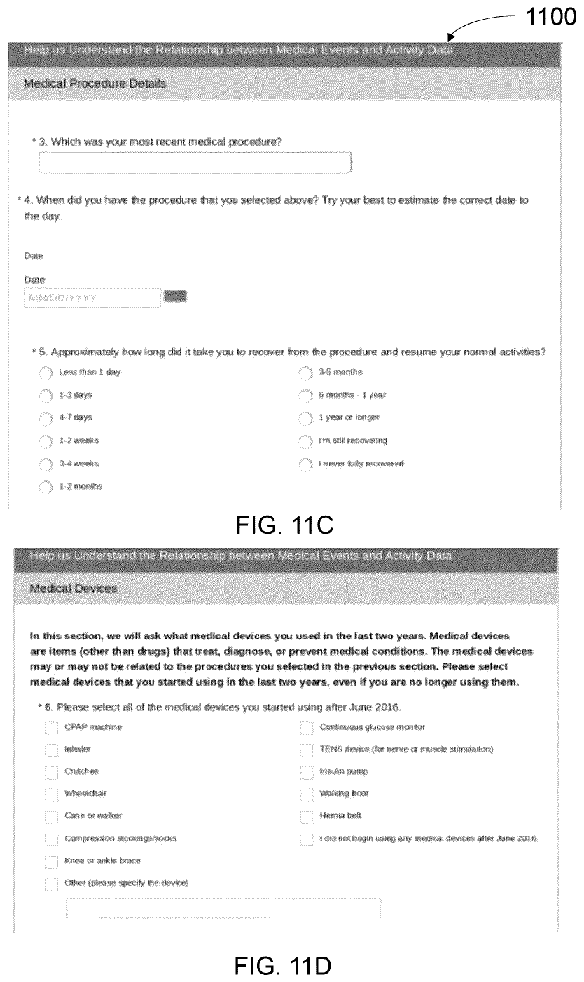

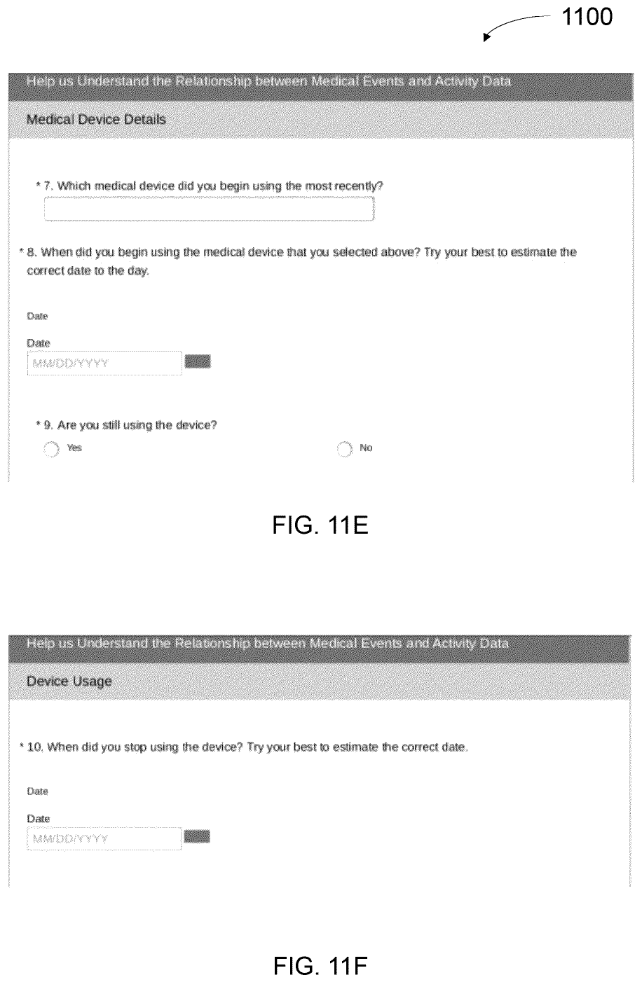

Supplementary Note 1: Survey (FIGS. 11A-F). FIGS. 11A-11F illustrates snapshots of the full survey deployed to users of the application. The survey asked about medical procedures the members have undergone in the 2 years prior to taking the survey.

Supplementary Note 2: Processing of steps, heart rate, and sleep data. Fitbit-collected data of steps, heart rate, and sleep were used to get daily aggregates of activity statistics. A part of daily activity features used in this work (sleep efficiency) were accessed from the public Fitbit application programming interface, whereas others were derived from the minute-level intraday activity data (total number of steps, fraction of minutes with >0 steps, maximum of 3- and 30-minute rolling steps sum, 95th percentile heart rate). Selected step daily features (total number of steps, maximum of 3-and 30-minute rolling steps sum) were winsorized at respective 0.999-th quantiles. The daily sleep efficiency feature, ranging originally 0-100 (mean=90.7, sd=11.4), was transformed with a log-based function to handle its high positive skewness, resulting in a (modified) efficiency feature ranging 0-100 (mean=56.8, sd=16.7).

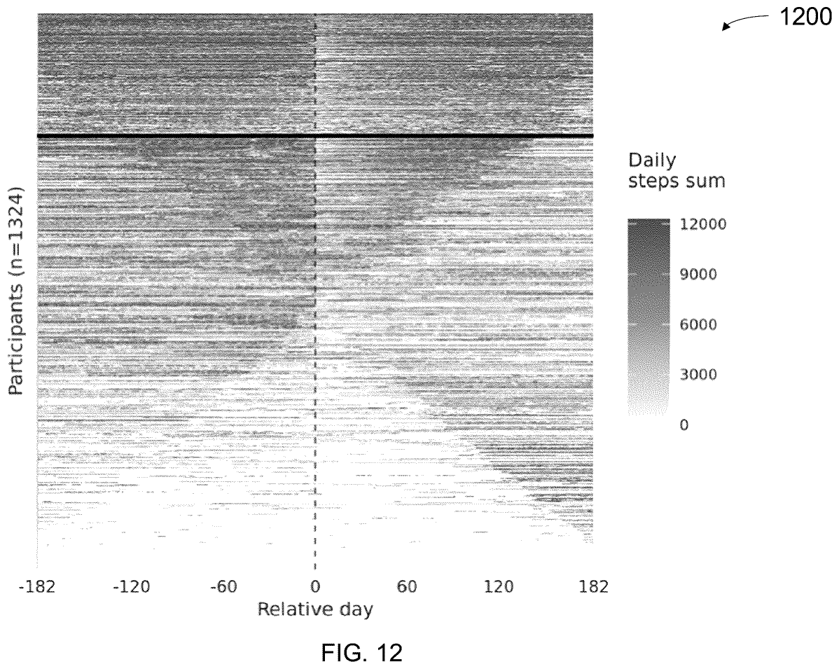

Supplementary Note 3: Data coverage (FIG. 12). FIG. 12 illustrates a plot showing step data coverage in a statistical analysis sample (n=1,324). The heatmap color corresponds to the daily number of steps (winsorized at 12,000 for visualization purposes) across days relative to self-reported surgery date (x-axis) in the observation window from 182 days before to 182 days after the surgery. The solid black horizontal line separates participants (n=295) who passed a step data density requirement for use in the experiment.

Supplementary Note 4: Data preparation for machine learning. To ensure maximal data quality in the reporting of surgery dates, cases with high likelihood of misreporting were systematically identified using a change point detection methodology:

At each time t of the daily number of steps time-series of length T, a four-piecewise function was fitted with the main change point located at t and the remaining function components optimized to minimize the fit residuals. The function's shape was restricted to represent expected post-surgery activity pattern, but flexible enough to account for various lengths of recovery and signal strength, or even no signal at all (see the figure below, part (a)). The likelihood Lt of the main change point being at time t was quantified by using a (standardized) difference between residuals e from single constant fit and residuals ut from fitted four-piecewise function with k parameters: Lt=(Σe2−Σut2)/(Σut2)/(T−k). In the figure below, part (b) shows an exemplary trajectory of Lt values for one participant. Time point t=tmax that maximizes Lt for a participant was defined as an algorithm-identified surgery date.

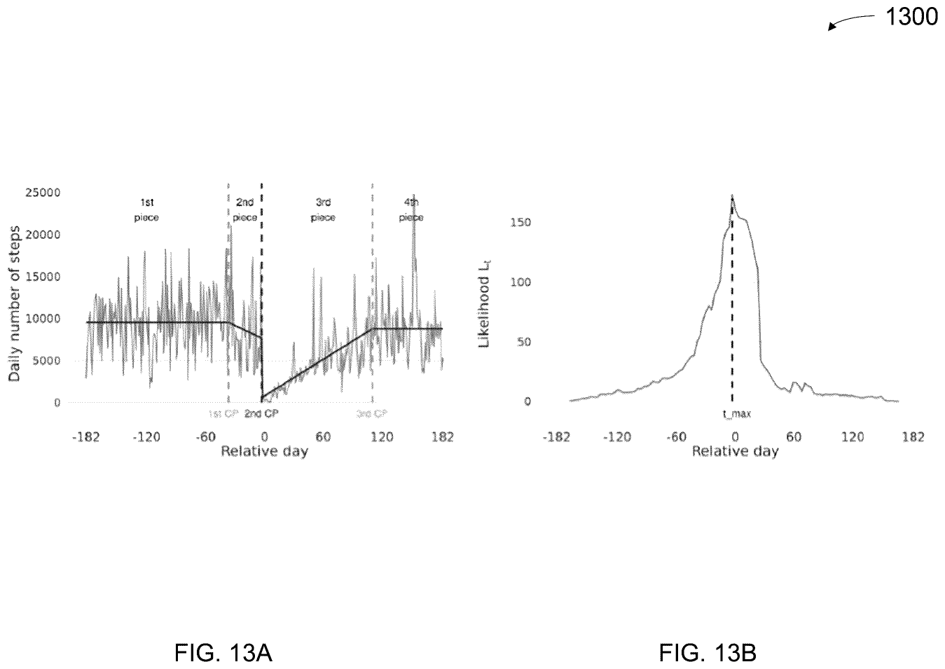

FIG. 13A provides an illustration of a four-piecewise fit used in change point (CP) detection procedure: (1) 1st piece: a constant, (2) 1st CP, (3) 2nd piece: a linear function with negative slope joined with Ist piece, or a constant same as Ist piece, (3) 2nd CP: the main CP located at a fixed time point t, (4) 3rd piece: a linear function with positive slope joined with 4th piece, or a constant same as 4th piece, (5) 3rd CP, (6) 4th piece: a constant. In the procedure, at each fixed time point t, the 2nd CP is fixed at t, and the remaining components of the four piecewise fit are optimized to reduce the fit residuals. FIG. 13B illustrates an exemplary trajectory of the likelihood Lt. Here, t=−3 maximizes Lt; the fit that corresponds to 2nd CP located at t=−3 is shown in figure left (a).

Finally, the maximum likelihood value, Lt_max, was used to propose a heuristic rule: (a) if best fit signal is strong (Lt_max statistic above 30) and self-reported and algorithm-identified surgery date are more than 28 days apart-a participant is filtered out; (b) otherwise—participant is kept. After applying the rule, n=217 out of 295 participants were kept. The figure below shows normalized likelihood trajectories, (Lt/Lt_max), together with algorithm-identified surgery date for kept and rejected participants.

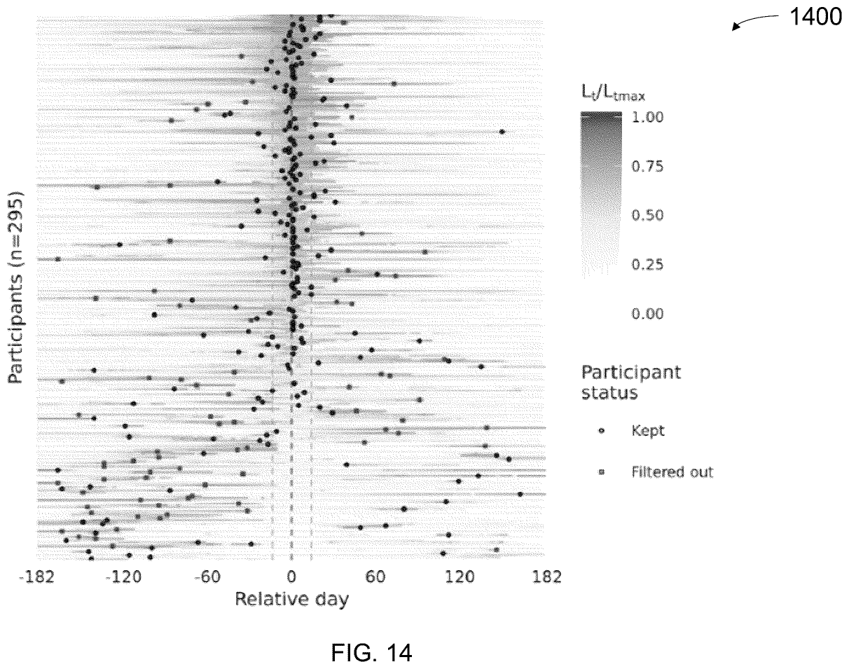

FIG. 14 illustrates a plot showing participants' (normalized) likelihood trajectories, (Lt/Lt_max), of the main change point being at time t across the observation window of 182 days before and 182 days after self-reported surgery time (x-axis).

Supplementary Note 5: Prediction of self-reported recovery times. The experiment was designed to evaluate performance of classifying self-reported recovery time labels. The model's performance was compared in six scenarios which differed in assumed availability of wearable PGHD: (1) no post-operative, no pre-operative, (2) no post-operative, 6 months (full) pre-operative, (3) 4 weeks post-operative, no pre-operative, (4) 4 weeks postoperative, 6 months pre-operative, (5) 6 months (full) post-operative, no pre-operative, (6) 6 months post-operative, 6months pre-operative; in each (1)-(6) case, demographics (age, gender) information was used.

Due to relatively small sample sizes for bone fracture and knee/hip replacement surgery predictive data sets (n=46 and 26, respectively; see Table 2), the experiment was narrowed down to analyzing the tendon or ligament surgery group only (n=125), and the machine learning task was cast as a binary classification of a participant into a faster (“0-2 months”) and slower (“>=3 months”) track of mobility recovery.

For each participant, a set of predictors was computed based on the four steps-derived daily measurements: total number of steps, fraction of minutes with non-zero steps count, number of steps in max of 3- and 30-minute rolling sum. The predictors were constructed as a measurement aggregate (median) over week(s) of time; the length of aggregation time period varied between one and 14 weeks long depending on distance from surgery date (the closer to the surgery, the higher resolution of the time periods). The aggregation of daily measures into irregular time periods was performed to avoid an extremely large ratio of number of predictors to number of observations while simultaneously making the most use of the data signal available.

In the notation used in this work, “relative week 0” always corresponds to a 7-day-long period that starts at the day of surgery, “relative week 1” lasts from the 7th to 13th day (inclusive) after the surgery, and “relative week-1” lasts from the 7th to 1st day (inclusive) before the surgery, etc. Then, activity measurements collected in relative weeks from −4 to 4 were aggregated over time periods of one week, activity measurements collected in relative weeks from −8 to −5 and from 5 to 8 were aggregated over time periods of two subsequent weeks, activity measurements collected in relative weeks from −12 to −9 and from 9 to 12 were aggregated over time periods of four subsequent weeks, activity measurements collected in relative weeks from −26 to −13 and from 13 to 26 were aggregated over time periods of fourteen weeks, respectively (the relative week 26 was exceptional as it consisted of 1 day only).

These predictors were further standardized to have mean 0 and variance 1 to avoid large differences in the order of values across predictors in the data set.

Also, to reflect participant's activity change w.r.t to the baseline (relative weeks from −26 to −13), additional predictors were defined as a ratio of (a) particular time period-aggregated value to (b) baseline weeks-aggregated value; these predictors were used in modeling only in the scenarios assuming pre-operative data is available. These variables were winsorized at value equal 3.

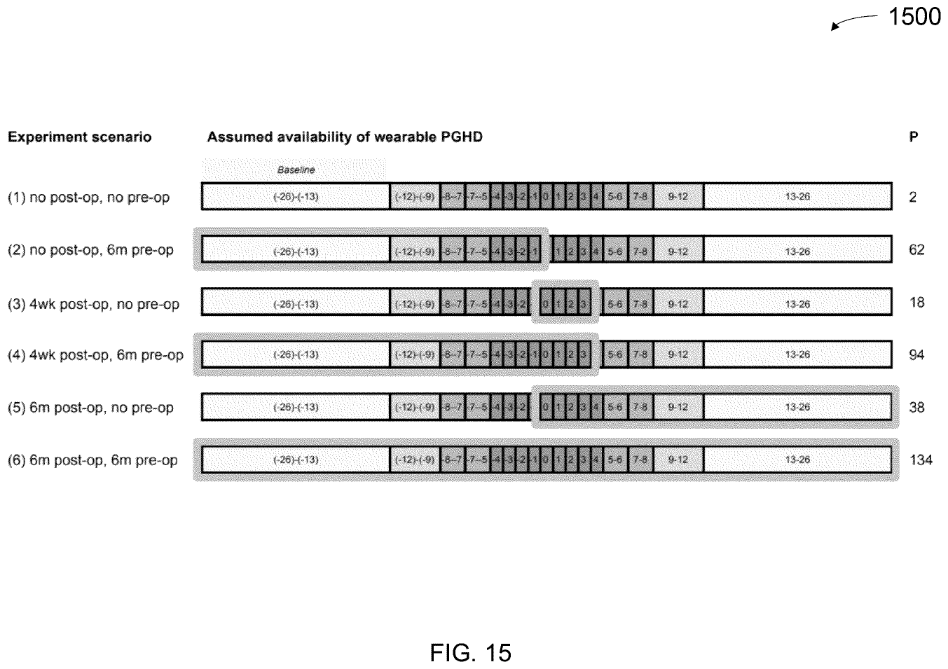

FIG. 15 shows assumed wearable PGHD availability in predictive modeling experiment scenarios (1)-(6). In each scenario, demographics (age, gender) were also used. The black rectangular box grid represents the grouping of relative week(s) into time periods for aggregation of daily activity measurements. The numbers within rectangular blocks denote a range of relative weeks within a certain aggregation time period. Green rectangular box is used to mark the weeks relative to the surgery from which wearable PGHD is assumed available in scenarios (2)-(6). The last column, “P,” summarizes the number of predictors (demographics and activity predictors combined) in each scenario.

The classification models were trained with the Extreme Gradient Boosting (XGBoost) algorithm. The choice of the algorithm was driven by its performance, ability to handle missing data, and interpretability of the results. A 100-repeat holdout procedure was used to estimate out-of-sample generalization of models' classification performance. In each of 100 repetitions, the dataset was split into training and test sets using an 80/20 split that was stratified by the outcome (faster, “0-2 months,” and slower, “>=3 months,” track of mobility recovery). Hyper-parameters were tuned on the training set by comparing AUROC predictive metric aggregated over 20 repetitions of 75/25 split stratified by the outcome; tuning was done by selecting the best combination of the following parameters: number of estimators, learning rate, maximum tree depth, gamma, minimum child weight, subsample proportion, out of 144 combinations considered. Then, the best parameters set was used to train the model on a full training set and to measure predictive performance on the holdout test sample. The predictive performance metric values (AUROC, AUPRC) summarized across 100 repetitions are reported.

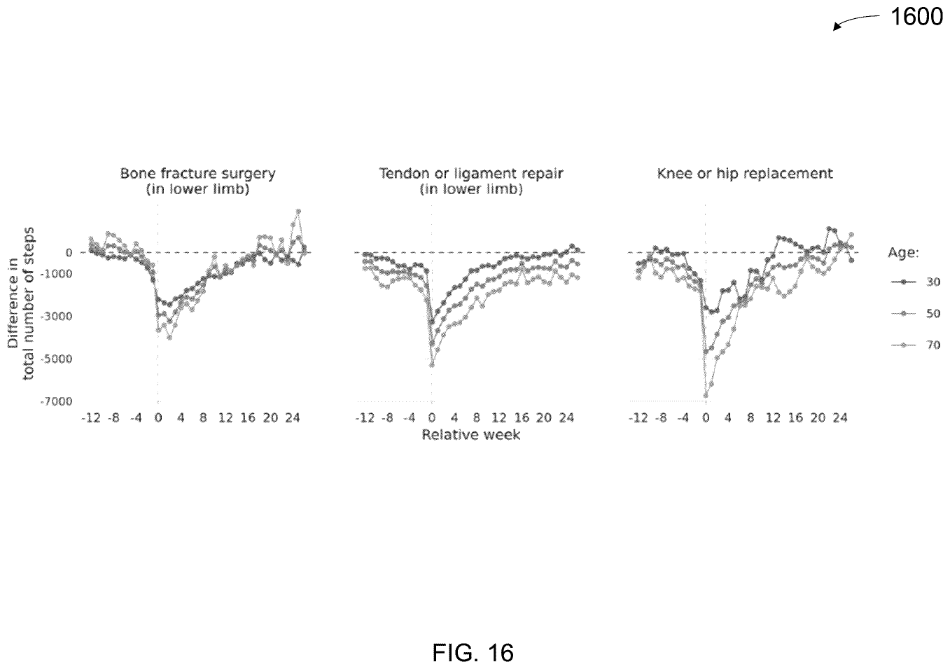

Supplementary Note 6: Impact of age on recovery trajectories. To demonstrate the validity of the cohort-level model, known effects due to age were explored. Increasing age is known to have a strong, negative influence on recovery timelines. The statistical model was therefore used to estimate average recovery trajectories at a range of ages (30, 50 and 70 years old), for an otherwise “typical” individual (female, with average baseline activity level among similar ones). The FIG. 16 describes fitted age-specific trajectories of daily number of steps across the three lower limb surgeries. Clearly, the age effect is demonstrated with higher difference in activity values after surgery compared to respective baseline levels. This effect is pronounced particularly strongly in knee/hip replacement sub-cohort in 1-5 weeks after the procedure; while it is not possible to determine the difference between the cases of knee and hip surgery based on the survey conducted, one can hypothesize that the values fitted for 70-years-old individual represent a higher proportion of hip replacement cases and correspond to a full/almost full immobilization days after the procedure.

FIG. 16 illustrates a daily total number of steps in subsequent weeks from 12 week before to 26 week after surgery compared to average value in the baseline period (weeks from 26 weeks before to 13 weeks before the surgery) for individuals at age 30, 50 and 70 and otherwise “typical” (female, with average baseline activity level among similar ones). Vertical plot panels correspond to three lower limb surgery types: bone fracture, tendon or ligament repair, and knee or hip replacement. The color of a point/line corresponds to the individual's age.

Supplementary Note 7: Trajectories of recovery across self-reported recovery time groups.

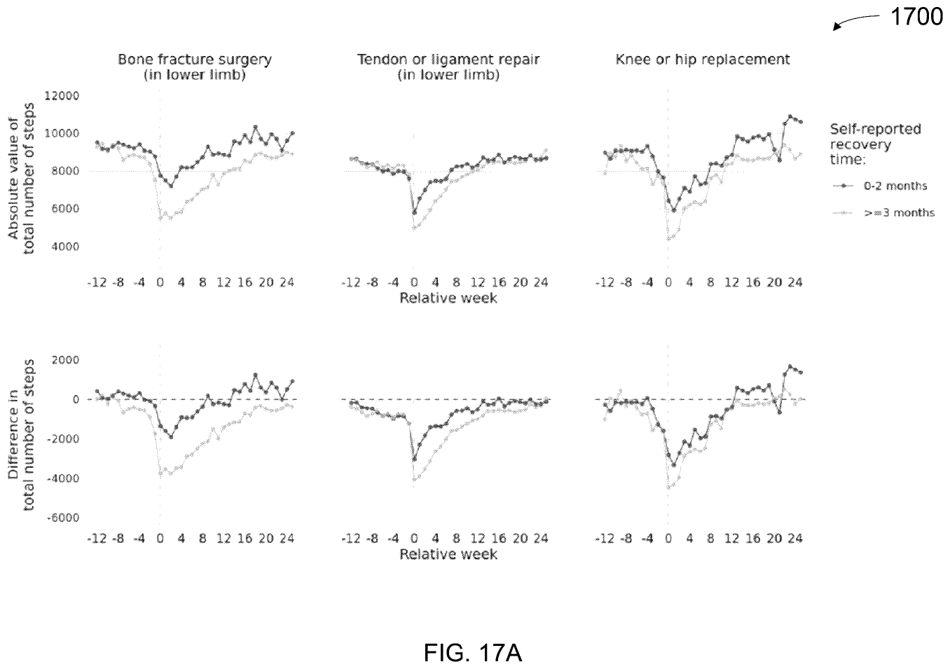

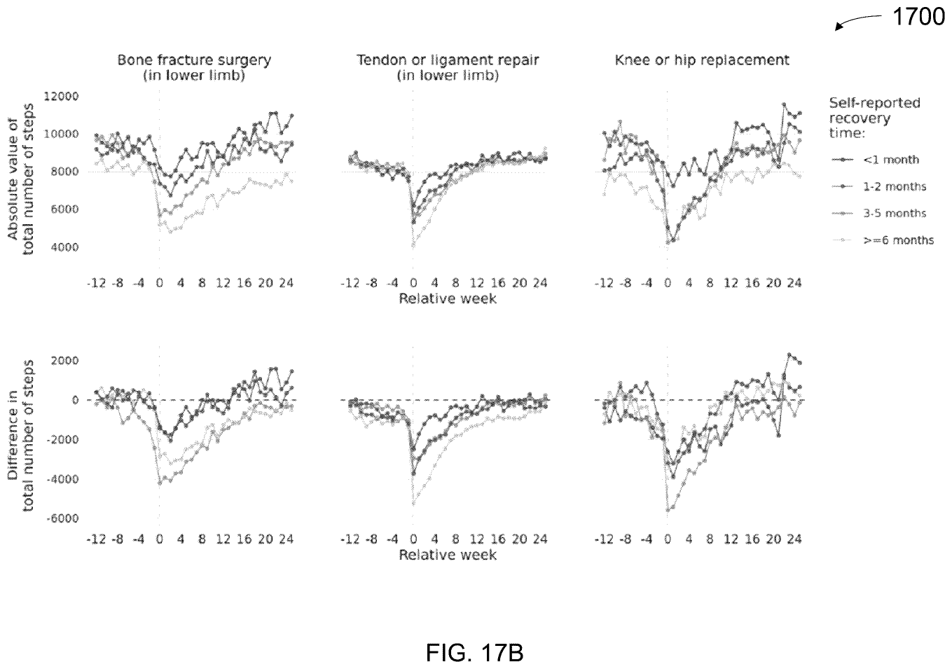

FIG. 17A shows a set of plots illustrating estimated trajectories of daily number of steps across two self-reported recovery time groups in subsequent weeks from week 12 before to week 26 after the surgery. The upper plots demonstrate absolute values of activity, the bottom plots demonstrate change with respect to the model-estimated baseline. Vertical plots correspond to three lower limb surgery types: bone fracture, tendon or ligament repair, and knee or hip replacement.

FIG. 17B shows a set of plots illustrating estimated trajectories of daily number of steps across four self-reported recovery time groups in subsequent weeks from week 12 before to week 26 after the surgery. The upper plots demonstrate absolute values of activity, the bottom plots panel—change with respect to the model-estimated baseline. Vertical plots correspond to three lower limb surgery types: bone fracture, tendon or ligament repair, and knee or hip replacement.

Supplementary note 8: Model-estimated average values of activity daily measurements

TABLE 4
Model-estimated average values of activity daily measurements (daily number of steps, 95th percentile
of heart rate (bpm), sleep efficiency) across three surgery type subcohorts (bone fracture repair,
tendon or ligament repair/reconstruction, knee or hip joint replacement) and across eight time
periods relative to self-reported surgery date: baseline and relative weeks −4, 0, 4, 8, 12,
16, 20. Relative week “0” was defined as a 7-day-long period that starts at the day of
surgery. Baseline was defined as relative weeks from −26 to −13. Showed are values estimated
for a “typical” cohort individual (female at age 40, with average baseline activity level
among otherwise similar ones) on a “typical” day (weekday, month of May).

| Activity | Surgery | | | | | | | | |
|---|---|---|---|---|---|---|---|---|---|
| daily | type | Time period relative to self-reported surgery date | | | | | | | |
| measurement | subcohort | Baseline | Week −4 | Week 0 | Week 4 | Week 8 | Week 12 | Week 16 | Week 20 |
| Number of | Bone frac. | 8900 | 8765 | 6315 | 6672 | 7512 | 8004 | 8618 | 8695 |
| steps | | | | | | | | | |
| Number of | Tendon | 8905 | 8003 | 5124 | 6823 | 7742 | 8095 | 8533 | 8478 |
| steps | | | | | | | | | |
| Number of | Knee/hip | 8815 | 8483 | 5179 | 6392 | 7632 | 8379 | 8718 | 8786 |
| steps | | | | | | | | | |
| 95th ptcl HR | Bone frac. | 103.9 | 103.4 | 100.1 | 101.7 | 102.9 | 102.1 | 102.8 | 103.0 |
| 95th ptcl HR | Tendon | 102.9 | 101.9 | 96.7 | 101.3 | 103.0 | 103.2 | 104.2 | 103.4 |
| 95th ptcl HR | Knee/hip | 103.8 | 103.1 | 98.8 | 102.8 | 104.2 | 104.7 | 105.2 | 104.9 |
| Sleep | Bone frac. | 60.4 | 59.7 | 58.8 | 58.8 | 58.8 | 59.3 | 58.9 | 58.4 |
| efficiency | | | | | | | | | |
| Sleep | Tendon | 57.6 | 57.4 | 56.4 | 56.6 | 56.1 | 56.9 | 57.3 | 57.4 |
| efficiency | | | | | | | | | |
| Sleep | Knee/hip | 57.7 | 58.1 | 56.1 | 55.6 | 55.0 | 57.4 | 56.1 | 55.5 |
| efficiency | | | | | | | | | |

TABLE 5
Model-estimated average values of activity daily measurement (daily number of steps) across three surgery type subcohorts
(bone fracture repair, tendon or ligament repair/reconstruction, knee or hip joint replacement), across three self-
reported recovery time groups (<1 month, 1-5 months, >=6 months), and across eight time periods relative to
self-reported surgery date: baseline and relative weeks −4, 0, 4, 8, 12, 16, 20. Relative week “0” was
defined as a 7-day-long period that starts at the day of surgery. Baseline was defined as relative weeks from −26
to −13. Showed are values estimated for a “typical” cohort individual (female at age 40, with average baseline
activity level among therwise similar ones) on a “typical” day (weekday, month of May).

| | | Self- | | | | | | | | | |
|---|---|---|---|---|---|---|---|---|---|---|---|
| Activity | Surgery | reported | | | | | | | | | |
| daily | type | recovery | | Time period relative to self-reported surgery date | | | | | | | |
| measurement | subcohort | time gr. | | Baseline | Week −4 | Week 0 | Week 4 | Week 8 | Week 12 | Week 16 | Week 20 |
| Number of | Bone | <1 | month | 9536 | 9679 | 8126 | 8726 | 9509 | 8848 | 10063 | 10108 |
| steps | frac. | | | | | | | | | | |
| Number of | Bone | 1-5 | months | 9386 | 9116 | 6561 | 7047 | 7937 | 8593 | 9462 | 9354 |
| steps | frac. | | | | | | | | | | |
| Number of | Bone | >=6 | months | 8027 | 8188 | 5192 | 5023 | 5783 | 6751 | 7158 | 7310 |
| steps | frac. | | | | | | | | | | |
| Number of | Tendon | <1 | month | 8676 | 7612 | 6189 | 7888 | 8380 | 8335 | 8821 | 8485 |
| steps | | | | | | | | | | | |
| Number of | Tendon | 1-5 | months | 8885 | 8182 | 5518 | 6891 | 7764 | 8182 | 8742 | 8786 |
| steps | | | | | | | | | | | |
| Number of | Tendon | >=6 | months | 9359 | 8099 | 4119 | 6017 | 7549 | 8059 | 8516 | 8408 |
| steps | | | | | | | | | | | |
| Number of | Knee/hip | <1 | month | 10542 | 9590 | 7829 | 7847 | 8987 | 9166 | 10391 | 9448 |
| steps | | | | | | | | | | | |
| Number of | Knee/hip | 1-5 | months | 9177 | 9152 | 4705 | 6185 | 7693 | 8880 | 9381 | 8966 |
| steps | | | | | | | | | | | |
| Number of | Knee/hip | >=6 | months | 7587 | 7221 | 4362 | 5866 | 7213 | 7579 | 7491 | 8436 |
| steps | | | | | | | | | | | |

Supplementary note 9: Model summary output. In statistical modeling of wearable PGHD, daily activity measurements were modeled with a linear mixed effect model (LMM), fitting a separate model (model 1) for each activity feature and surgery type subcohort. To further estimate trajectories of activity across time of recovery groups, the statistical model was extended by considering variables for self-reported recovery time groups (model 2—“extended”). Below we define LMM formula notation (common for both model 1 and model 2—“extended”) and report elements of LMM fit summary—variance and correlation components and coefficient estimates—for model 1 and model 2—“extended,” respectively. For the sake of space, we limit the report to one activity feature (daily sum of steps) and one surgery type subcohort (bone fracture surgery).

LMM formula notation:

The LMM formulas and LMM fit summary elements presented below share the following notation for data variables.
y—Numeric variable. Participant-and day-specific activity measurement.
time_indic—Factor variable. A week relative to a self-reported surgery date. Takes values: {baseline, −12, −11, . . . , −1, 0, 1, . . . , 26}, where “baseline” is set as reference factor level. Relative week “0” is a 7 days-long time period starting on a self-reported surgery day. “Baseline” is a time period defined as weeks from 26 week before to 13 week before the surgery.
age_centered—Numeric variable. Participant's age, centered at 40 (has value 0 for a 40 years old participant.
gender—Factor variable. Self-reported participant gender. Takes values:
{female, male, other}, where “female” is set as reference factor level.
date_isweekend—Factor variable. Flag whether or not a participant- and day-specific activity measurement was collected on a weekend day. Takes values: {0,1}, where “0” is set as reference factor level.
date_years_month—Factor variable. Label for a month of a year. Takes values: {Jan, Feb, . . . , Nov, Dec}, where “May” is set as reference factor level.
user_id—Factor variable. Participant-specific ID.
recovery_gr-Factor variable. Label for self-reported recovery time groups. Takes values: (a) {0-2 months, >=3 months}, where “0-2 months” is set as reference factor level, or (b) {<1 month, 1-5 months, >=6 months}, where “1-5 months” is set as reference factor level, or (c) {<1 month, 1-2 months, 3-5 months, >=6 months} where “1-2 months” is set as reference factor level.
   Linear mixed effect model 1:

$$y ∼ time\_indic * age\_centered + gender + date\_isweekend + {date\_years}_{-} month + ( 1 + date\_isweekend \vert user\_id )$$

| Groups | Name | Std. Dev. | Corr. |
|---|---|---|---|
| user_id | (Intercept) | 3444.2 | |
| | date_isweekend | 2043.2 | −0.438 |
| Residual | | 4085.2 | |

Linear mixed effect model 2—“extended”:

$$y ∼ time\_indic * age\_centered + time\_indic * recovery\_gr + age\_centered \text{} + gender + date \_isweekend + \\ date \_years \_month + ( 1 + date \_isweekend \vert user \_id )$$

| Groups | Name | Std. Dev. | Corr. |
|---|---|---|---|
| user_id | (Intercept) | 3540.8 | |
| | date_isweekend | 2126.4 | −0.495 |
| Residual | | 4150.7 | |

### Computer Systems

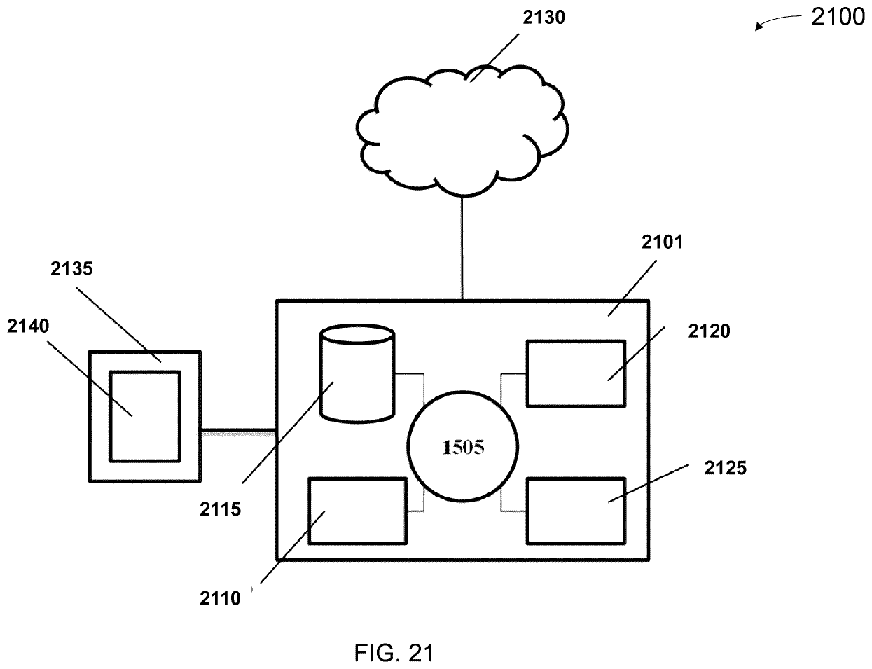

The present disclosure provides computer systems that are programmed to implement methods of the disclosure. FIG. 21 shows a computer system 2101 that is programmed or otherwise configured to predict time to recovery from wearable sensor data. The computer system 2101 can regulate various aspects of predicting time to recovery of the present disclosure, such as, for example, implementing one or more machine learning algorithms. The computer system 2101 can be an electronic device of a user or a computer system that is remotely located with respect to the electronic device. The electronic device can be a mobile electronic device.

The computer system 2101 includes a central processing unit (CPU, also “processor” and “computer processor” herein) 2105, which can be a single core or multi core processor, or a plurality of processors for parallel processing. The computer system 1501 also includes memory or memory location 2110 (e.g., random-access memory, read-only memory, flash memory), electronic storage unit 2115 (e.g., hard disk), communication interface 1520 (e.g., network adapter) for communicating with one or more other systems, and peripheral devices 2125, such as cache, other memory, data storage and/or electronic display adapters. The memory 2110, storage unit 2115, interface 2120 and peripheral devices 2125 are in communication with the CPU 2105 through a communication bus (solid lines), such as a motherboard. The storage unit 2115 can be a data storage unit (or data repository) for storing data. The computer system 2101 can be operatively coupled to a computer network (“network”) 2130 with the aid of the communication interface 2120. The network 2130 can be the Internet, an internet and/or extranet, or an intranet and/or extranet that is in communication with the Internet. The network 2130 in some cases is a telecommunication and/or data network. The network 2130 can include one or more computer servers, which can enable distributed computing, such as cloud computing. The network 2130, in some cases with the aid of the computer system 2101, can implement a peer-to-peer network, which may enable devices coupled to the computer system 2101 to behave as a client or a server.

The CPU 2105 can execute a sequence of machine-readable instructions, which can be embodied in a program or software. The instructions may be stored in a memory location, such as the memory 2110. The instructions can be directed to the CPU 2105, which can subsequently program or otherwise configure the CPU 2105 to implement methods of the present disclosure. Examples of operations performed by the CPU 2105 can include fetch, decode, execute, and writeback.

The CPU 2105 can be part of a circuit, such as an integrated circuit. One or more other components of the system 2101 can be included in the circuit. In some cases, the circuit is an application specific integrated circuit (ASIC).

The storage unit 2115 can store files, such as drivers, libraries and saved programs. The storage unit 2115 can store user data, e.g., user preferences and user programs. The computer system 2101 in some cases can include one or more additional data storage units that are external to the computer system 2101, such as located on a remote server that is in communication with the computer system 2101 through an intranet or the Internet.

The computer system 2101 can communicate with one or more remote computer systems through the network 2130. For instance, the computer system 2101 can communicate with a remote computer system of a user (e.g., a mobile device). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 2101 via the network 2130.

Methods as described herein can be implemented by way of machine (e.g., computer processor) executable code stored on an electronic storage location of the computer system 2101, such as, for example, on the memory 2110 or electronic storage unit 2115. The machine executable or machine readable code can be provided in the form of software. During use, the code can be executed by the processor 2105. In some cases, the code can be retrieved from the storage unit 2115 and stored on the memory 2110 for ready access by the processor 2105. In some situations, the electronic storage unit 2115 can be precluded, and machine-executable instructions are stored on memory 2110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2101 can include or be in communication with an electronic display 2135 that comprises a user interface (UI) 2140 for providing, for example, a recovery score. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2105. The algorithm can, for example, predict a time to recovery.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### CONCLUSION

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A method for performing a health intervention, comprising:
(a) obtaining, from a wearable sensor, health data of a user, wherein the health data comprises a time series stream of health events of the user;
(b) determining a trajectory of the user in a latent health space based at least in part on the time series stream of the health events of the user;
(c) standardizing the health data of the user to generate standardized health data of the target user, wherein said standardizing comprising transforming the health data based at least in part on one or more attributes of the wearable sensor;
(d) selecting a health intervention for the user based at least in part on the trajectory of the user in the latent health space; and
(e) causing initiation of the health intervention on behalf of the user via transmitting a notification to a user device of the target user, wherein the user device comprises the wearable sensor.

2. The method of claim 1, wherein the health data of the user comprises one or both of behavior data or medical data.

3. The method of claim 1, wherein the time series stream of the health events of the user comprises a plurality of physical statistics data of the user over a plurality of time periods.

4. The method of claim 1, wherein the user device comprises a wrist-adapter to reversibly attach to the wrist of the target user.

5. A method for training a machine learning prediction system, comprising:
(a) accessing, by the machine learning prediction system, a set of training data for a plurality of users of a population, the training data representative of physical statistics and symptoms for the plurality of users for each of a plurality of time periods, the training data collected by a wearable sensor;
(b) training, by the machine learning prediction system, a machine learning model using the accessed set of training data, the machine learning model configured to predict, for a first acute health condition, acute health condition onset for a user based on physical statistics of the user, the physical statistics comprising data corresponding to (i) a weather condition corresponding to the user, (ii) a planned event corresponding to the user, and a (iii) a geographical location corresponding to the user; and
(c) in response to determining, via the machine learning model, a probability of acute health condition onset for a user exceeds a threshold, performing one or more intervention actions on behalf of the target user.

6. The method of claim 5, wherein the machine learning model comprises one or more of a decision tree algorithm or a random forest.

7. The method of claim 5, wherein the physical statistics of the user are obtained via a smartwatch worn by the user.

8. A method comprising:
(a) obtaining wearable sensor data comprising a plurality of time series measurements collected from a target user during a first time period; and
(b) predicting, via a machine learning model, a recovery trajectory from an acute condition or debilitating event at least in part by processing the wearable sensor data comprising the plurality of time series measurements from the first time period, wherein the classifier is trained at least in part by:
using a set of training data from a first population cohort comprising a plurality of users, the set of training data comprising a confirmed case of the acute condition or debilitating event of a first user of the plurality of users and wearable sensor data from the plurality of users, wearable sensor data from the first user of the plurality of users comprising physical statistics and symptoms collected over a plurality of time periods and associated with the acute condition or debilitating event, wherein at least one of the consecutive time periods is prior to an onset of the acute condition or debilitating event and at least one of the consecutive time periods is after the onset of the acute condition or debilitating event, wherein the first user is placed in the first population cohort based at least in part on a pattern of the wearable sensor data from the first user.

9. The method of claim 8, wherein the time series measurements correspond to at least one of sleep efficiency, step count, or heart rate.

10. The method of claim 8, wherein the wherein the time series measurements correspond to at least two of sleep efficiency, step count, and heart rate.

11. The method of claim 8, wherein the wearable sensor data is collected daily throughout the first time period.

12. The method of claim 8, wherein the machine learning model comprises a classifier.

13. The method of claim 8, wherein the classifier comprises one or more of a decision tree algorithm or a random forest.

14. The method of claim 8, further comprising initiating a health intervention on behalf of the target user.

15. The method of claim 14, wherein initiating the health intervention further comprises causing modification of the interface displayed by the user device of the target user to display a notification configured to change a behavior of the target user.

16. The method of claim 8, wherein initiating the health intervention further comprises causing a test kit corresponding to the acute health condition to be sent to the target user

17. The method of claim 8, wherein the acute or debilitating event is a surgery.

18. A system comprising:
one or more processors; and
one or more memories storing computer-executable instructions that, when executed, cause the one or more processors to perform operations comprising:
(a) obtaining wearable sensor data comprising a plurality of time series measurements collected from a target user during a first time period; and
(b) predicting, via a machine learning model, a recovery trajectory from an acute condition or debilitating event at least in part by processing the wearable sensor data comprising the plurality of time series measurements from the first time period, wherein the classifier is trained at least in part by:
using a set of training data from a first population cohort comprising a plurality of users, the set of training data comprising a confirmed case of the acute condition or debilitating event of a first user of the plurality of users and wearable sensor data from the plurality of users, wearable sensor data from the first user of the plurality of users comprising physical statistics and symptoms collected over a plurality of time periods and associated with the acute condition or debilitating event, wherein at least one of the consecutive time periods is prior to an onset of the acute condition or debilitating event and at least one of the consecutive time periods is after the onset of the acute condition or debilitating event, wherein the first user is placed in the first population cohort based at least in part on a pattern of the wearable sensor data from the first user.

19. The system of claim 18, wherein the time series measurements correspond to at least one of sleep efficiency, step count, or heart rate.

20. The system of claim 18, wherein the wherein the time series measurements correspond to at least two of sleep efficiency, step count, and heart rate.

21. The system of claim 18, wherein the wearable sensor data is collected daily throughout the first time period.

22. The system of claim 18, wherein the machine learning model comprises a classifier.

23. The system of claim 18, wherein the classifier comprises one or more of a decision tree algorithm or a random forest.

24. The system of claim 18, further comprising initiating a health intervention on behalf of the target user.

25. The system of claim 24, wherein initiating the health intervention further comprises causing modification of the interface displayed by the user device of the target user to display a notification configured to change a behavior of the target user.

26. The system of claim 18, wherein initiating the health intervention further comprises causing a test kit corresponding to the acute health condition to be sent to the target user.
